SEQUENCE LISTING (6 sequences)

SEQ ID NO: 1 - protein, 287 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLEDPSAVPVFSVDYGELDFQWREKTPEPPVPCVPEQTEYATIVFPSGMGTSSPARRGSADGPRSAQPLRPEDGHCSWPL

SEQ ID NO: 2 - DNA, 2111 bp, Homo sapiens
acagtttcccttccgctcacctccgcctgagcagtggagaaggcggcactctggtggggctgctccaggcatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctccacctttacacatgcccaggcagcacctcaggccctttgtggggcagggaagctgaggcagtaagcgggcaggcagagctggaggcctttcaggcccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgccgtggcctatccactcctcacatccacacactgcacccccactcctggggcagggccaccagcatccaggcggccagcaggcacctgagtggctgggacaagggatcccccttccctgtggttctattatattataattataattaaatatgagagcatgctaagga

SEQ ID NO: 3 - protein, 504 aa, Homo sapiens
MKEESAAQLGCCHRPMALGGTGGSLSPSLDFQLFRGDQVFSACRPLPDMVDAHGPSCASWLCPLPLAPGRSALLACLQDLDLNLCTPQPAPLGTDLQGLQEDALSMKHEPPGLQASSTDDKKFTVKYPQNKDKLGKQPERAGEGAPCPAFSSHNSSSPPPLQNRKSPSPLAFCPCPPVNSISKELPFLLHAFYPGYPLLLPPPHLFTYGALPSDQCPHLLMLPQDPSYPTMAMPSLLMMVNELGHPSARWETLLPYPGAFQASGQALPSQARNPGAGAAPTDSPGLERGGMASPAKRVPLSSQTGTAALPYPLKKKNGKILYECNICGKSFGQLSNLKVHLRVHSGERPFQCALCQKSFTQLAHLQKHHLVHTGERPHKCSVCHKRFSSSSNLKTHLRLHSGARPFQCSVCRSRFTQHIHLKLHHRLHAPQPCGLVHTQLPLASLACLAQWHQGALDLMAVASEKHMGYDIDEVKVSSTSQGKARAVSLSSAGTPLVMGQDQNN

SEQ ID NO: 4 - DNA, 1687 bp, Homo sapiens
actttgtcccacatgccctggggaggtagcacaaagggaggaccatggtcttctaactgaggaacctgagcagatgccttgacccagaccagagacccaagccaaggaaagcccatgatcaccagacaggtaatggggatatgaaggaagaatcagctgcacaattaggttgttgtcataggcccatggccctgggaggtacagggggctccctgtcccccagcctggacttccagctcttccgaggtgaccaggtcttctcagcctgcagaccacttccagacatggtggatgctcatggcccatcctgtgccagctggctgtgtcccttgcccctggcaccgggcaggtctgcactgctggcctgcctacaggacctggacctgaacctgtgcaccccacagccggcacccctgggcacagacctgcagggcctccaagaggacgccttgagcatgaagcacgagccaccagggctgcaggccagctccaccgatgacaagaaattcacagtcaagtacccacagaacaaggacaagctgggaaaacagccagaaagagctggcgagggggccccctgcccagccttctcctctcataacagctcttccccaccaccgctgcagaacagaaagagccccagccccttggctttctgcccctgtccccctgtcaactccatctccaaggagctcccatttctcctccacgccttctaccctggatacccacttctcctgcctccaccccacctgttcacctatggggccctaccttctgaccaatgtccccacctcctcatgctgccccaagacccctcctaccccaccatggctatgcctagcctgctgatgatggtcaatgagctggggcaccccagcgctcggtgggagaccctgcttccctacccaggggccttccaagcctctggccaagctctgccttcccaggcccgaaatccaggtgctggagctgccccaaccgactccccaggcctggagcgtggtggcatggcatctccagcaaagcgggtcccattgagttcccagacaggcaccgcagccttgccttacccgctgaaaaagaagaatggcaaaatcctgtacgagtgcaacatatgtggcaagagctttgggcagctctccaatctcaaggtccacctgcgtgtgcacagtggagagcgtccattccagtgtgccttgtgccagaagagcttcactcaacttgcccacctgcagaagcaccacctggtgcacactggggagcggccccacaagtgctcggtgtgccacaagcgcttcagcagctccagtaacctcaagacccacctgcgcctgcactccggggcccggcccttccagtgcagtgtctgccggagtcgcttcacccagcacatccacctgaagctgcaccatcggctgcatgccccacagccctgtggcctggtgcacacccagctgcccctggcctctctggcctgccttgcccaatggcaccagggggcactagatcttatggcggtggcatctgagaaacacatgggctatgacatagatgaggtcaaagtgtcctcgacatcccaggggaaagcaagagcagtgagcctgagcagtgccgggactcccctggtgatggggcaggaccagaacaattaaaaatgtttcttctgtcaaaaaaaaaaaaaaaa

SEQ ID NO: 5 - protein, 504 aa, Homo sapiens; identical to SEQ ID NO: 3

SEQ ID NO: 6 - DNA, 4076 bp, Homo sapiens
ctcttcttcttaaacaaaccacaaacggatgtgagggaaggaaggtgtttcttttactcctgagcccagacacctcactctgttccgtctaagcttgttttgctgaacacttttttttaaaaaaggaaaaagaaaaggagttgcttgatgtgagagtgaaatggacgtaagattttatccacctccagcccagcccgccgctgcgcccgacgctccctgtctgggaccttctccctgcctggacccctactattgcaacaagtttgacggtgagaacatgtatatgagcatgacagagccgagccaggactatgtgccagccagccagtcctaccctggtccaagcctggaaagtgaagacttcaacattccaccaattactcctccttccctcccagaccactcgctggtgcacctgaatgaagttgagtctggttaccattctctgtgtcaccccatgaaccataatggcctgctaccatttcatccacaaaacatggacctccctgaaatcacagtctccaatatgctgggccaggatggaacactgctttctaattccatttctgtgatgccagatatacgaaacccagaaggaactcagtacagttcccatcctcagatggcagccatgagaccaaggggccagcctgcagacatcaggcagcagccaggaatgatgccacatggccagctgactaccattaaccagtcacagctaagtgctcaacttggtttgaatatgggaggaagcaatgttccccacaactcaccatctccacctggaagcaagtctgcaactccttcaccatccagttcagtgcatgaagatgaaggcgatgatacctctaagatcaatggtggagagaagcggcctgcctctgatatggggaaaaaaccaaaaactcccaaaaagaagaagaagaaggatcccaatgagccccagaagcctgtgtctgcctatgcgttattctttcgtgatactcaggccgccatcaagggccaaaatccaaacgctacctttggcgaagtctctaaaattgtggcttcaatgtgggacggtttaggagaagagcaaaaacaggtctataaaaagaaaaccgaggctgcgaagaaggagtacctgaagcaactcgcagcatacagagccagccttgtatccaagagctacagtgaacctgttgacgtgaagacatctcaacctcctcagctgatcaattcgaagccgtcggtgttccatgggcccagccaggcccactcggccctgtacctaagttcccactatcaccaacaaccgggaatgaatcctcacctaactgccatgcatcctagtctccccaggaacatagcccccaagccgaataaccaaatgccagtgactgtctctatagcaaacatggctgtgtcccctcctcctcccctccagatcagcccgcctcttcaccagcatctcaacatgcagcagcaccagccgctcaccatgcagcagccccttgggaaccagctccccatgcaggtccagtctgccttacactcacccaccatgcagcaaggatttactcttcaacccgactatcagactattatcaatcctacatctacagctgcacaagttgtcacccaggcaatggagtatgtgcgttcggggtgcagaaatcctcccccacaaccggtggactggaataacgactactgcagtagtgggggcatgcagagggacaaagcactgtaccttacttgagaatctgaacacctcttctttccactgaggaattcagggaagtgttttcaccatggattgctttgtacagtcaaggcagttctccattttattagaaaatacaagttgctaagcacttaggaccatttgagcttgtgggtcacccactctggaagaaatagtcatgcttctttattatttttttaatcctttatggacattgtttttcttcttccctgaaggaaatttggaccattcagattttatgttggttttttgctgtgaagtgctgcgctctagtaactgccttagcaactgtagatgtctcggataaaagtcctggattttccattggttttcataatgggtgtttatatgaaactactaaagactttttaaatggcttgatgtagcagtcatagcaagtttgtaaatagcatctatgttacactctcctagagtataaaatgtgaatgtttttgtagctaaattgtaattgaaactggctcattccagtttattgatttcacaataggggttaaattggcaaacattcatatttttacttcatttttaaaacaactgactgatagttctatattttcaaaatatttgaaaataaaaagtattcccaagtgattttaatttaaaaacaaattggctttgtctcattgatcagacaaaaagaaactagtattaagggaagcgcaaacacatttattttgtactgcagaaaaattgcttttttgtatcactttttgtgtaatggttagtaaatgtcatttaagtccttttatgtataaaactgccaaatgcttacctggtattttattagatgcagaaacagattggaaacagctaaattacaacttttacatatggctctgtcttattgtttcttcatactgtgtctgtatttaatctttttttatggaacctgttgcgcctatttatgaaataataaatataggtgtttgtaagtaaatttgttagtatttgaaagaggtttctttgatgttttaacttttgctggcaaaaaaaaattcacgcttggtgtgaatactttattatttagtttttacagtaacatgaataaagccaaacctgcttttcatttagcagcaaattaaagtaaccagtccttatttctgcatttctttggttgatgcaaacaaaaaactattatatttaagaactttatttcttcatacgacataacagaattgccctccaagtcacacaagctccaagactaaacaaacagacaggtcctctgtcttaaaaaggttacttcttggttctcagctggttctagtcaattctgaaccaccaccccccgccccccgcaaaaaagtaaaagtcaaaccaaacttcctcaagctgcatgcttttcacaaaatccagaaagcatttaagaattgaactaggggctggaagaagtgaaagggaagcatctaaaaatgaaaggtgagtaaccagatagcaaaagaaaagggaaagccatccaaatttgaaagctgttgatagaaattgagattcttgctgtcttttgtgcctctacaagctactactcattccagaattcctgggtcttccaagaggattcttaaggtaccagagatttgctagggaaccaaaagtgcttgagaatctgcctgagggcttgcatagctttcacattaaaaaaagaaaaagctagcagatttactcctttttaggggatcatatcaagaaagttagtctggttggaaaccaagagaatggctgatgtctctttcttggaatatgtgaaataaatttagcagtttaactaaatacaaatatatgcattgtgtaatccactcagaattaaacagacaaaaggtatgcttgctttggaatgattttaggcattgtacaaccttgaatcacttgagcatgtaataactaataaataatgcagatccatgtgattattaaaatgactgtagctgagagctctaattttcctgtcttgaaactgtataagaactcatgtgattaagttcacagtttattgtttgtctgtttagtattttagaaatataccagcactactaattaactaatgtcttttatttattatattatgataaagtaaaaatttcacttgcattaagtctaaactgagaaggtaattactgggaggagaatgagcagctttgactttgacaggcggtttgtgcaggaaagcacagtgccgtgttgtttacagcttttctagagcagctgtgcgaccagggtagagagtgttgaaattcaataccaaatacagtaaaaacaaatgtaaataaaagaaaacacatcatcaataaaactgttattatgcgtgaccgta

(19) United States
(12) Patent Application Publication
(10) Pub. No.: US 2022/0298580 A1
(43) Pub. Date: Sep. 22, 2022

(54) IMMUNE SIGNATURES PREDICTIVE OF RESPONSE TO PD-1 BLOCKADE IN RICHTER'S TRANSFORMATION

(71) Applicant: DANA-FARBER CANCER INSTITUTE, INC., Boston, MA (US)

(72) Inventors: Catherine Wu, Brookline, MA (US); Erin Parry, Westwood, MA (US)

(73) Assignee: DANA-FARBER CANCER INSTITUTE, INC., Boston, MA (US)

(21) Appl. No.: 17/634,465

(22) PCT Filed: Aug. 27, 2020
(86) PCT No.: PCT/US2020/048192

§ 371 Date: Feb. 10, 2022

Related U.S. Application Data
(60) Provisional application No. 62/893,987, filed on Aug. 30, 2019.

(51) Int. Cl. C12Q 1/6886; A61P 35/00; C07K 16/28

(52) U.S. Cl. CPC C12Q 1/6886; A61P 35/00; C07K 16/2818; C12Q 2600/112; C12Q 2600/178

(57) ABSTRACT

The present invention relates to methods for identifying Richter's transformation patients who benefit from PD-1 blockade.

### RELATED APPLICATIONS

This application claims the benefit of priority under 35 U.S.C. § 119(e) to U.S. Provisional Application No. 62/893,987, filed Aug. 30, 2019, which is incorporated herein by reference in its entirety.

### GOVERNMENT LICENSE RIGHTS

This invention was made with government support under grant numbers P01 CA206978 and R01 CA155010 awarded by The National Institutes of Health. The Government has certain rights in the invention.

### SEQUENCE LISTING

The instant application contains a Sequence Listing which has been submitted electronically in ASCHII format and is hereby incorporated by reference in its entirety. Said ASCII copy, created on Aug. 27, 2020, is named 52095-6340001WO_ST25.txt and is 21.6 KB bytes in size.

### BACKGROUND OF THE INVENTION

Richter's transformation (RT), also known as Richter syndrome (RS), refers to the transformation of B cell chronic lymphocytic leukemia (CLL) into an aggressive lymphoma, most commonly diffuse large B-cell lymphoma (DLBCL). The prognosis for RT is poor. Median survival time is less than one year. Recent results demonstrate that RT, but not the underlying CLL, responds to programmed cell death protein-1 (PD-1) checkpoint blockade with an overall response rate of 43%-65%. A pressing need exists to identify predictors of response and resistance to PD-1 checkpoint blockade in RT.

### SUMMARY OF THE INVENTION

The present invention is based upon the surprising discovery that ZNF683 (Hobit) marks a T cell effector/effector memory population associated with effective anti-tumor immunity following programmed cell death protein-1 (PD-1) checkpoint blockade for Richter's transformation. Distinct T cell populations and gene signatures associated with response to PD-1 checkpoint blockade (CPB) through examination of a discovery cohort of serial bone marrow samples collected from RT patients treated with nivolumab (PD-1 CPB) plus ibrutinib on clinical trial (NCT 02420912) were identified using single-cell RNA-sequencing. As described herein, these signatures genes, that are present at baseline, are predictive of response and are used to identify patients, prior to treatment, who would benefit from PD-1 blockade.

As described in detail below, the gene expression signature predicts clinical response and resistance to PD-1 blockade, i.e., PD-1 inhibition, with, e.g., nivolumab, in patients with RT.

Methods of determining whether PD-1 inhibition (checkpoint blockade) in a subject, e.g., a human subject, with Richter's transformation will result in clinical benefit in the subject are provided. Specifically, provided herein are methods of treating a subject with RT. The methods are carried out by obtaining a test sample from a subject having or at risk of RT; determining the expression level of at least one RT-associated gene in the test sample; comparing the expression level of the RT-associated gene in the test sample with the expression level of the RT-associated gene in a reference sample; determining whether programmed cell death protein-1 (PD-1) checkpoint blockade will inhibit RT and provide a clinical benefit to the subject if the expression level of the RT-associated gene in the test sample is differentially expressed as compared to the level of the RT-associated gene in the reference sample; and administering an effective amount of a PD-1 inhibitor to the subject in whom the expression level of the RT-associated gene in the test sample is differentially expressed as compared to the level of the RT-associated gene in the reference sample.

For example, the test sample is obtained from bone marrow, a tumor tissue, a tumor microenvironment, a plasma sample, or a blood sample.

For example, clinical benefit in the subject comprises complete or partial response as defined by response evaluation criteria in solid tumors (RECIST), stable disease as defined by RECIST, or long-term survival in spite of disease progression or response as defined by irRC criteria.

In some cases, the test sample is obtained from the subject with RT, wherein the RT-associated gene comprises Zinc finger protein 683 gene (ZNF683); and it is determined that inhibition of PD-1 in the subject with RT will result in clinical benefit in the subject if the expression level of the ZNF683 gene in the test sample is higher than the level of the ZNF683 gene in the reference sample.

In other cases, the test sample is obtained from the subject with RT, wherein the RT-associated gene comprises a thymocyte selection-associated high mobility group box protein gene (TOX); and it is determined that inhibition of PD-1 in the subject with RT will not result in clinical benefit in the subject if the expression level of the TOX gene in the test sample is higher than the level of the TOX gene in the reference sample. In some cases, the methods further comprise administering an inhibitor of the RT-associated gene, e.g., a TOX gene, with a higher level of expression compared to the level of the RT-associated gene in the reference sample, thereby treating the RT. For example, the inhibitor of the RT-associated gene comprises a small molecule inhibitor, RNA interference (RNAi), microRNA (miRNA), an antibody, an antibody fragment, an antibody drug conjugate, or any combination thereof. In some cases, a PD-1 inhibitor is administered in conjunction with the inhibitor of the RT-associated gene.

In some cases, the methods also include treating the subject with a chemotherapeutic agent, radiation therapy, cryotherapy, hormone therapy, or immunotherapy. For example, the chemotherapeutic agent comprises dacarbazine, temozolomide, nab-paclitaxel, paclitaxel, cisplatin, or carboplatin.

In one aspect, the inhibitor of PD-1 comprises a small molecule inhibitor, RNA interference (RNAi), microRNA (miRNA), an antibody, an antibody fragment, an antibody drug conjugate, an aptamer, a chimeric antigen receptor (CAR), a T cell receptor, or any combination thereof. Preferably, the small molecule inhibitor comprises a selective small molecule inhibitor.

For example, the PD-1 inhibitor comprises an antibody or antibody fragment, wherein the antibody or antibody fragment comprises nivolumab, cemiplimab, pembrolizumab, avelumab, atezolizumab, or durvalumab. In some cases, the antibody or antibody fragment is partially humanized, fully humanized, or chimeric.

In some cases, the PD-1 inhibitor comprises a small molecule. For example, the small molecule comprises ibrutinib.

In one aspect, the reference sample is obtained from healthy normal tissue, cancer that received a clinical benefit from PD-1 inhibition, or cancer that did not receive a clinical benefit from PD-1 inhibition. In some cases, the reference sample is obtained from healthy normal tissue from the same individual as the test sample or one or more healthy normal tissues from different individuals.

In some cases, the expression level of the RT-associated gene is detected via a Gene Hybridization Array, or a real time reverse transcriptase polymerase chain reaction (real time RT-PCR) assay.

Preferably, tumor cell survival, tumor cell proliferation, or tumor metastasis is inhibited, e.g., by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%.

In some cases, tumor cell cytokinesis is inhibited, e.g., by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%.

In one aspect, tumor cell growth is reduced, e.g., by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%

In some cases, the methods further comprise administering to the subject a chemotherapeutic agent, radiation therapy, cryotherapy, hormone therapy, or immunotherapy. For example, the chemotherapeutic agent comprises thalidomide, lenalidomide, ibrutinib, ixazomib, bortezomib, carfilzomib, melphalan, vincristine, cyclophosphamide, doxorubicin, liposomal doxorubicin, or bendamustine.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

The phrase “aberrant expression” is used to refer to an expression level that deviates from (i.e., an increased or decreased expression level) the normal reference expression level of the gene.

By “agent” is meant any small compound, antibody, nucleic acid molecule, or polypeptide, or fragments thereof.

By “alteration” is meant a change (increase or decrease) in the expression levels or activity of a gene or polypeptide as detected by standard art-known methods such as those described herein. As used herein, an alteration includes at least a 1% change in expression levels, e.g., at least a 2%, 3%, 4%, 5%, 6%, 7%, 8%, 9%, 10%, 20%, 30%, 40%, 50%, 60%, 70%, 80%, 90%, or 100% change in expression levels. For example, an alteration includes at least a 5%-10% change in expression levels, preferably a 25% change, more preferably a 40% change, and most preferably a 50% or greater change in expression levels.

The term “antibody” (Ab) as used herein includes monoclonal antibodies, polyclonal antibodies, multispecific antibodies (e.g., bispecific antibodies), and antibody fragments, as long as they exhibit the desired biological activity. The term “immunoglobulin” (Ig) is used interchangeably with “antibody” herein.

By “binding to” a molecule is meant having a physicochemical affinity for that molecule.

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g., β-galactosidase or luciferase). Depending on the method used for detection, the amount and measurement of the change can vary. Determination of statistical significance is within the ability of those skilled in the art, e.g., the number of standard deviations from the mean that constitute a positive result.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to ameliorate the symptoms of a disease, e.g., RT, relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The term “expression profile” is used broadly to include a genomic expression profile. Profiles may be generated by any convenient means for determining a level of a nucleic acid sequence, e.g., quantitative hybridization of microRNA, labeled microRNA, amplified microRNA, complementary/synthetic DNA (cDNA), etc., quantitative polymerase chain reaction (PCR), and ELISA for quantitation, and allow the analysis of differential gene expression between two samples. A subject or patient tumor sample is assayed. Samples are collected by any convenient method, as known in the art. According to some embodiments, the term “expression profile” means measuring the relative abundance of the nucleic acid sequences in the measured samples.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity, e.g., at least 80%, at least 85%, at least 90%, at least 95%, or at least 99% identity. Polynucleotides having “substantial identity” to an endogenous sequence are typically capable of hybridizing with at least one strand of a double-stranded nucleic acid molecule.

As used herein, “obtaining” as in “obtaining an agent” includes synthesizing, purchasing, or otherwise acquiring the agent.

Unless specifically stated or obvious from context, as used herein, the term “or” is understood to be inclusive. Unless specifically stated or obvious from context, as used herein, the terms “a”, “an”, and “the” are understood to be singular or plural.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier should be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

By “protein” or “polypeptide” or “peptide” is meant any chain of more than two natural or unnatural amino acids, regardless of post-translational modification (e.g., glycosylation or phosphorylation), constituting all or part of a naturally-occurring or non-naturally occurring polypeptide or peptide, as is described herein.

The terms “preventing” and “prevention” refer to the administration of an agent or composition to a clinically asymptomatic individual who is at risk of developing, susceptible, or predisposed to a particular adverse condition, disorder, or disease, and thus relates to the prevention of the occurrence of symptoms and/or their underlying cause.

The term “prognosis,” “staging,” and “determination of aggressiveness” are defined herein as the prediction of the degree of severity of the neoplasia, e.g., RT, and of its evolution as well as the prospect of recovery as anticipated from usual course of the disease. Once the aggressiveness has been determined, appropriate methods of treatments are chosen.

The terms “short hairpin RNA” or “shRNA” are used interchangeably herein refer to is an artificial RNA molecule with a tight hairpin turn that can be used to silence target gene expression via RNA interference (RNAi). Expression of shRNA in cells is typically accomplished by delivery of plasmids or through viral or bacterial vectors. shRNA is an advantageous mediator of RNAi in that it has a relatively low rate of degradation and turnover.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 10%, 25%, 50%, 75%, or 100%.

The term “sample” as used herein refers to a biological sample obtained for the purpose of evaluation in vitro. Exemplary tissue samples for the methods described herein include tissue samples from RT tumors or the surrounding microenvironment (i.e., the stroma). With regard to the methods disclosed herein, the sample or patient sample preferably may comprise any body fluid or tissue. In some embodiments, the bodily fluid includes, but is not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of a blood sample, a plasma sample, a serum sample, and a urine sample. In exemplary aspects, the sample comprises blood or a fraction thereof (e.g., plasma or serum). Preferred samples are whole blood, serum, plasma, or urine. A sample can also be a partially purified fraction of a tissue or bodily fluid.

A reference sample can be a “normal” sample, from a donor not having the disease or condition fluid, or from a normal tissue in a subject having the disease or condition. A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested or at the start of a prospective study.

By “specifically binds” is meant a compound or antibody that recognizes and binds a polypeptide of the invention, but which does not substantially recognize and bind other molecules in a sample, for example, a biological sample, which naturally includes a polypeptide of the invention.

By “selective inhibitor” is meant a compound or substrate that selectively binds a target protein to ultimately reduce or eliminate the protein's activity.

The term “subject” as used herein includes all members of the animal kingdom prone to suffering from the indicated disorder. In some aspects, the subject is a mammal, and in some aspects, the subject is a human. The methods are also applicable to companion animals such as dogs and cats as well as livestock such as cows, horses, sheep, goats, pigs, and other domesticated and wild animals.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with cancer (e.g., RT) is within the ability of those in the art. Subjects suffering from, and suspected of suffering from, a specific disease, condition, or syndrome are not necessarily two distinct groups.

As used herein, “susceptible to” or “prone to” or “predisposed to” or “at risk of developing” a specific disease or condition refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. It will be appreciated that, although not precluded, treating a disorder or condition does not require that the disorder, condition or symptoms associated therewith be completely eliminated.

In some cases, a composition of the invention is administered orally or systemically. Other modes of administration include rectal, topical, intraocular, buccal, intravaginal, intracistemal, intracerebroventricular, intratracheal, nasal, transdermal, within/on implants, or parenteral routes. The term “parenteral” includes subcutaneous, intrathecal, intravenous, intramuscular, intraperitoneal, or infusion. Intravenous or intramuscular routes are not particularly suitable for long-term therapy and prophylaxis. They could, however, be preferred in emergency situations. Compositions comprising a composition of the invention can be added to a physiological fluid, such as blood. Oral administration can be preferred for prophylactic treatment because of the convenience to the patient as well as the dosing schedule. Parenteral modalities (subcutaneous or intravenous) may be preferable for more acute illness, or for therapy in patients that are unable to tolerate enteral administration due to gastrointestinal intolerance, ileus, or other concomitants of critical illness. Inhaled therapy may be most appropriate for pulmonary vascular diseases (e.g., pulmonary hypertension).

Pharmaceutical compositions may be assembled into kits or pharmaceutical systems for use in arresting cell cycle in rapidly dividing cells, e.g., cancer cells. Kits or pharmaceutical systems according to this aspect of the invention comprise a carrier means, such as a box, carton, tube, having in close confinement therein one or more container means, such as vials, tubes, ampoules, bottles, syringes, or bags. The kits or pharmaceutical systems of the invention may also comprise associated instructions for using the kit.

Any compositions or methods provided herein can be combined with one or more of any of the other compositions and methods provided herein.

Where applicable or not specifically disclaimed, any one of the embodiments described herein are contemplated to be able to combine with any other one or more embodiments, even though the embodiments are described under different aspects of the invention.

These and other embodiments are disclosed and/or encompassed by, the following Detailed Description.

### BRIEF DESCRIPTION OF THE DRAWINGS

FIG. 1A-FIG. 1B are a series diagrams and graphs showing single-cell RNA-sequencing cohort for determinants of response to programmed cell death protein 1 (PD-1) blockade. FIG. 1A is a swimmer's plot showing the 8 patients included in the discovery cohort. Richter's transformation (RT) responders are represented in purple, RT non-responders are represented in blue and relapsed/refractory chronic lymphocytic leukemia (CLL) patients in salmon. Panel B shows the validation cohort of RT patients. FIG. 1B is an experimental schema showing flow cytometry sorting strategy of populations used for single-cell RNA-sequencing experiments followed by analytic strategy and joint clustering of immune and tumor cell populations.

FIG. 2A-FIG. 2E are a series of graphs and a heat showing that Expression changes in T cell populations related to response status. FIG. 2A is joint graph LargeVis embedding of immune cell populations with subsequent sub-clustering of T and natural killer (NK) cells revealing 11 distinct transcriptional clusters. FIG. 2B is a heat map showing marker genes and cluster identities for 11 identified T and NK cell clusters. FIG. 2C is a joint graph showing differences between cells from RT responders and RT non-responders. FIG. 2D is a set of bar graphs showing population distributions across serial marrow samples. FIG. 2E is a set of graphs showing the distribution of T and NK cell populations in RT and CLL marrows as compared to normal marrows.

FIG. 3A-FIG. 3C are a set of plots and heat maps showing that gene expression changes associated with PD-1 response. FIG. 3A is a set of volcano plots showing gene expression differences comparing all baseline and month 3 samples from RT responders to all samples from RT non-responders for Clusters 1 (E/EM), 8 (Exhausted), 2 (CD4) and 9 (Cytotoxic CD4). FIG. 3B is a set of heat maps with accompanying graphs showing quantitative cell proportion differences and qualitative gene expression changes between RT responders and non-responders. FIG. 3C is a set of graphs showing cells in ZNF683 expressing cluster 1, normalized counts of cells with positive ZNF683 expression and percent of cells in cluster 1 expressing ZNF683 for serial samples from RT responders and RT non-responders.

FIG. 4A-FIG. 4E are a set of diagrams and graphs showing clonal expansion of T cells with tumor specificity in RT responders. FIG. 4A is a diagram showing the prevalence of top clonotypes as determined by single-cell T-cell receptor (TCR) sequencing. FIG. 4B is a set of T-distributed Stochastic Neighbor Embedding (t-SNE) diagrams showing the distribution of top 2 enriched clonotypes across T cell clusters. FIG. 4C is a set of graphs showing a cluster distribution of top 10 enriched TCRs at post PD-1 time point in pre and post PD-1 samples. P<0.001 by poisson proportion test. FIG. 4D is a diagram showing that enriched clonotypes are detected in peripheral blood samples. FIG. 4E is a set of T-distributed Stochastic Neighbor Embedding (t-SNE) diagrams showing that cloned TCRs show RT tumor specificity.

FIG. 5A-FIG. 5H are a set of graphs showing the single-cell RNA-sequencing metrics. FIG. 5A is a graph showing the number of cells per sample for immune samples. FIG. 5B is a graph showing the number of cells per sample for tumor samples. FIG. 5C is a graph showing the number of unique molecular identifiers (UMIs) per cell for immune samples. FIG. 5D is a graph showing the number of unique molecular identifiers (UMIs) per cell for tumor samples. FIG. 5E is a graph showing the number of genes per cell for immune samples. FIG. 5F is a graph showing the number of gene per cell for tumor samples. FIG. 5G is a graph showing mitochondrial content prior to filtering for immune samples. FIG. 5H is a graph showing mitochondrial content prior to filtering for tumor samples.

FIG. 6A-FIG. 6D are a set of graphs and diagrams showing a joint clustering of immune cells. FIG. 6A is a Conos joint graph of non-tumor clusters. FIG. 6B is a joint graph colored by sample and sample type. FIG. 6C is a set of t-SNE diagrams showing cluster distributions for each sample. FIG. 6D shows marker gene panels for joint graph highlighting major cell types.

FIG. 7A-FIG. 7E is a series of images showing sub-clustering of lymphocytes.

### DETAILED DESCRIPTION OF THE INVENTION

The present invention is based upon the surprising discovery that ZNF683 (Hobit) marks a T cell effector/effector memory population associated with effective anti-tumor immunity following programmed cell death protein-1 (PD-1) checkpoint blockade for Richter's transformation (RT). Distinct T cell populations and gene signatures associated with response to PD-1 checkpoint blockade (CPB) through examination of a discovery cohort of serial bone marrow samples collected from RT patients treated with nivolumab (PD-1 CPB) plus ibrutinib on clinical trial (NCT 02420912) were identified using single-cell RNA-sequencing. As described herein, these signatures genes, that are present at baseline, are predictive of response and are used to identify patients, prior to treatment, who benefit from PD-1 blockade.

The introduction of immune checkpoint blockade has transformed the clinical practice of oncology. Hodgkin's lymphoma (HL), for example, has exemplified the dramatic responses achievable with PD-1 blockade, and has been attributed to 9p24.1 (PD-L1/PD-L2 locus) genetic alterations. However, across hematologic malignancies, the types of responses observed in HL have proven to be the exception rather than the rule, with generally disappointing activity for cancers such as chronic lymphocytic leukemia (CLL), myeloma and follicular lymphoma. In this context, the responses of RT to PD-1 blockade, recently reported across several clinical trials at 43-65%, have been unexpected. Described as a transformation of indolent CLL into an aggressive lymphoma, RT has historically resulted in dismal overall survival and poor responsiveness to chemotherapy. Thus, its relative responsiveness to PD-1 blockade opens new therapeutic avenues for this once near-fatal condition. Described herein is a better understanding of why this particular malignancy possesses greater activity against PD-1 blockade compared to other blood malignancies.

Transcription factor, Zinc finger protein 683 (ZNF683), was identified to mark a population enriched in responders, both at baseline and time of response, and validated this an independent cohort of RT patients. It was demonstrated that enriched T cell clones that comprise the ZNF683 high effector/effector memory state additionally exist in transitional and memory states. CD8 E/EM T cells of RT non-responders showed increased expression of TOX, a recently uncovered master regulator of cell exhaustion (padj=0.00016). This cell subtype in RT responders upregulated a contrasting program of activating transcription factors as well as the co-stimulatory gene CD226 (padj=0.04). Through TCR cloning and functional testing, ZNF683 overexpression and cytotoxicity assays, ZNF683 was demonstrated to mediate T cell state and anti-tumor response.

Richter's Transformation (RT)

Richter's transformation (RT), also known as Richter syndrome (RS), refers to the transformation of B cell chronic lymphocytic leukemia (CLL) and/or small lymphocytic lymphoma (SLL) into an aggressive lymphoma, most commonly diffuse large B-cell lymphoma (DLBCL). RT occurs in approximately 2-10% of all CLL/SLL patients during the course of their disease. The most common symptoms of RT arise from a sudden and dramatic increase in the size of lymph nodes (also known as ‘lymphadenopathy’) characterized by usually painless areas of swelling in the neck, axilla, abdomen (most commonly in the spleen, also known as ‘splenomegaly’) or groin. The prognosis for RT is poor with a median survival time of less than one year. Recent results demonstrate that RT, but not the underlying CLL, responds to PD-1 checkpoint blockade with an overall response rate of 43%-65%.

Programmed Cell Death Protein-1 (PD-1)

PD-1, also known as cluster of differentiation 279 (CD279), is a member of the CD28 superfamily. It delivers negative signals upon interaction with its two ligands, PD-L1 or PD-L2. PD-1 and its ligands are broadly expressed and exert a wider range of immunoregulatory roles in T cell activation and tolerance compared with other CD28 members. PD-1 and its ligands are also involved in attenuating infectious immunity and tumor immunity, and facilitating chronic infection and tumor progression (Jin et. al, (2011), Curr Top Microbiol Immunol. 350:17-37).

The PD-1 protein in humans is encoded by the PDCDJ gene. PD-1 is a cell surface receptor that belongs to the immunoglobulin superfamily and is expressed on T cells and pro-B cells.

An exemplary PD-1 amino acid sequence is provided at NCBI Accession No. XP_016859782, version XP_016859782.1, incorporated herein by reference and set forth below (SEQ ID NO: 1):

| | |
|---|---|
| 1 | mqipqapwpv vwavlqlgwr pgwfldspdr pwnpptfspa |
| | llvvtegdna tftcsfsnts |
| 61 | esfvlnwyrm spsnqtdkla afpedrsqpg qdcrfrvtql |
| | pngrdfhmsv vrarrndsgt |
| 121 | ylcgaislap kaqikeslra elrvterrae vptahpspsp |
| | rpagqfqtlv vgvvggllgs |
| 181 | lvllvwvlav icsraargti garrtgqple dpsavpvfsv |
| | dygeldfqwr ektpeppvpc |
| 241 | vpeqteyati vfpsgmgtss parrgsadgp rsaqplrped |
| | ghcswpl |

An exemplary PD-1 nucleic acid sequence is provided at NCBI Accession No. XM_017004293, version XM_017004293.1, incorporated herein by reference and set forth below (SEQ ID NO: 2):

| | |
|---|---|
| 1 | acagtttccc ttccgctcac ctccgcctga gcagtggaga |
| | aggcggcact ctggtggggc |
| 61 | tgctccaggc atgcagatcc cacaggcgcc ctggccagtc |
| | gtctgggcgg tgctacaact |
| 121 | gggctggcgg ccaggatggt tcttagactc cccagacagg |
| | ccctggaacc cccccacctt |
| 181 | ctccccagcc ctgctcgtgg tgaccgaagg ggacaacgcc |
| | accttcacct gcagcttctc |
| 241 | caacacatcg gagagcttcg tgctaaactg gtaccgcatg |
| | agccccagca accagacgga |
| 301 | caagctggcc gccttccccg aggaccgcag ccagcccggc |
| | caggactgcc gcttccgtgt |
| 361 | cacacaactg cccaacgggc gtgacttcca catgagcgtg |
| | gtcagggccc ggcgcaatga |
| 421 | cagcggcacc tacctctgtg gggccatctc cctggccccc |
| | aaggcgcaga tcaaagagag |
| 481 | cctgcgggca gagctcaggg tgacagagag aagggcagaa |
| | gtgcccacag cccaccccag |
| 541 | cccctcaccc aggccagccg gccagttcca aaccctggtg |
| | gttggtgtcg tgggcggcct |
| 601 | gctgggcagc ctggtgctgc tagtctgggt cctggccgtc |
| | atctgctccc gggccgcacg |
| 661 | agggacaata ggagccaggc gcaccggcca gcccctggag |
| | gacccctcag ccgtgcctgt |
| 721 | gttctctgtg gactatgggg agctggattt ccagtggcga |
| | gagaagaccc cggagccccc |
| 781 | cgtgccctgt gtccctgagc agacggagta tgccaccatt |
| | gtctttccta gcggaatggg |
| 841 | cacctcatcc cccgcccgca ggggctcagc tgacggccct |
| | cggagtgccc agccactgag |
| 901 | gcctgaggat ggacactgct cttggcccct ctgaccggct |
| | tccttggcca ccagtgttct |
| 961 | gcagaccctc caccatgagc ccgggtcagc gcatttcctc |
| | aggagaagca ggcagggtgc |
| 1021 | aggccattgc aggccgtcca ggggctgagc tgcctggggg |
| | cgaccggggc tccagcctgc |
| 1081 | acctgcacca ggcacagccc caccacagga ctcatgtctc |
| | aatgcccaca gtgagcccag |
| 1141 | gcagcaggtg tcaccgtccc ctacagggag ggccagatgc |
| | agtcactgct tcaggtcctg |
| 1201 | ccagcacaga gctgcctgcg tccagctccc tgaatctctg |
| | ctgctgctgc tgctgctgct |
| 1261 | gctgctgcct gcggcccggg gctgaaggcg ccgtggccct |
| | gcctgacgcc ccggagcctc |
| 1321 | ctgcctgaac ttgggggctg gttggagatg gccttggagc |
| | agccaaggtg cccctggcag |
| 1381 | tggcatcccg aaacgccctg gacgcagggc ccaagactgg |
| | gcacaggagt gggaggtaca |
| 1441 | tggggctggg gactccccag gagttatctg ctccctgcag |
| | gcctagagaa gtttcaggga |
| 1501 | aggtcagaag agctcctggc tgtggtgggc agggcaggaa |
| | acccctccac ctttacacat |
| 1561 | gcccaggcag cacctcaggc cctttgtggg gcagggaagc |
| | tgaggcagta agcgggcagg |
| 1621 | cagagctgga ggcctttcag gcccagccag cactctggcc |
| | tcctgccgcc gcattccacc |
| 1681 | ccagcccctc acaccactcg ggagagggac atcctacggt |
| | cccaaggtca ggagggcagg |
| 1741 | gctggggttg actcaggccc ctcccagctg tggccacctg |
| | ggtgttggga gggcagaagt |
| 1801 | gcaggcacct agggcccccc atgtgcccac cctgggagct |
| | ctccttggaa cccattcctg |
| 1861 | aaattattta aaggggttgg ccgggctccc accagggcct |
| | gggtgggaag gtacaggcgt |
| 1921 | tcccccgggg cctagtaccc ccgccgtggc ctatccactc |
| | ctcacatcca cacactgcac |
| 1981 | ccccactcct ggggcagggc caccagcatc caggcggcca |
| | gcaggcacct gagtggctgg |
| 2041 | gacaagggat cccccttccc tgtggttcta ttatattata |
| | attataatta aatatgagag |
| 2101 | catgctaagg a |

Zinc Finger Protein 683. Hobit (ZNF683)

ZNF683, Hobit, is known to be a regulator of T cell residency in both mouse and human. While T cell expression of ZNF683 in mouse is restricted to the resident memory program, the pattern of ZNF683 expression has previously been demonstrated to be different in humans. Specifically, it regulates long-lived effector cells in CMV infection that retain immediate effector functions upon stimulation.

An exemplary ZNF683 amino acid sequence is provided at NCBI Accession No. NP_001108231, version NP_001108231.1, incorporated herein by reference and set forth below (SEQ ID NO: 3):

| | |
|---|---|
| 1 | mkeesaaqlg cchrpmalgg tggslspsld fqlfrgdqvf |
| | sacrplpdmv dahgpscasw |
| 61 | lcplplapgr sallaclqdl dlnlctpqpa plgtdlqglq |
| | edalsmkhep pglqasstdd |
| 121 | kkftvkypqn kdklgkqper agegapcpaf sshnsssppp |
| | lqnrkspspl afcpcppvns |
| 181 | iskelpfllh afypgyplll ppphlftyga lpsdqcphll |
| | mlpqdpsypt mampsllmmv |
| 241 | nelghpsarw etllpypgaf qasgqalpsq arnpgagaap |
| | tdspglergg maspakrvpl |
| 301 | ssqtgtaalp yplkkkngki lyecnicgks fgqlsnlkvh |
| | lrvhsgerpf qcalcqksft |
| 361 | qlahlqkhhl vhtgerphkc svchkrfsss snlkthlrlh |
| | sgarpfqcsv crsrftqhih |
| 421 | lklhhrlhap qpcglvhtql plaslaclaq whqgaldlma |
| | vasekhmgyd idevkvssts |
| 481 | qgkaravsls sagtplvmgq dqnn |

An exemplary ZNF683 nucleic acid sequence is provided at NCBI Accession No. NM_001114759, version NM_001114759.2, incorporated herein by reference and set forth below (SEQ ID NO: 4):

| | |
|---|---|
| 1 | actttgtccc acatgccctg gggaggtagc acaaagggag |
| | gaccatggtc ttctaactga |
| 61 | ggaacctgag cagatgcctt gacccagacc agagacccaa |
| | gccaaggaaa gcccatgatc |
| 121 | accagacagg taatggggat atgaaggaag aatcagctgc |
| | acaattaggt tgttgtcata |
| 181 | ggcccatggc cctgggaggt acagggggct ccctgtcccc |
| | cagcctggac ttccagctct |
| 241 | tccgaggtga ccaggtcttc tcagcctgca gaccacttcc |
| | agacatggtg gatgctcatg |
| 301 | gcccatcctg tgccagctgg ctgtgtccct tgcccctggc |
| | accgggcagg tctgcactgc |
| 361 | tggcctgcct acaggacctg gacctgaacc tgtgcacccc |
| | acagccggca cccctgggca |
| 421 | cagacctgca gggcctccaa gaggacgcct tgagcatgaa |
| | gcacgagcca ccagggctgc |
| 481 | aggccagctc caccgatgac aagaaattca cagtcaagta |
| | cccacagaac aaggacaagc |
| 541 | tgggaaaaca gccagaaaga gctggcgagg gggccccctg |
| | cccagccttc tcctctcata |
| 601 | acagctcttc cccaccaccg ctgcagaaca gaaagagccc |
| | cagccccttg gctttctgcc |
| 661 | cctgtccccc tgtcaactcc atctccaagg agctcccatt |
| | tctcctccac gccttctacc |
| 721 | ctggataccc acttctcctg cctccacccc acctgttcac |
| | ctatggggcc ctaccttctg |
| 781 | accaatgtcc ccacctcctc atgctgcccc aagacccctc |
| | ctaccccacc atggctatgc |
| 841 | ctagcctgct gatgatggtc aatgagctgg ggcaccccag |
| | cgctcggtgg gagaccctgc |
| 901 | ttccctaccc aggggccttc caagcctctg gccaagctct |
| | gccttcccag gcccgaaatc |
| 961 | caggtgctgg agctgcccca accgactccc caggcctgga |
| | gcgtggtggc atggcatctc |
| 1021 | cagcaaagcg ggtcccattg agttcccaga caggcaccgc |
| | agccttgcct tacccgctga |
| 1081 | aaaagaagaa tggcaaaatc ctgtacgagt gcaacatatg |
| | tggcaagagc tttgggcagc |
| 1141 | tctccaatct caaggtccac ctgcgtgtgc acagtggaga |
| | gcgtccattc cagtgtgcct |
| 1201 | tgtgccagaa gagcttcact caacttgccc acctgcagaa |
| | gcaccacctg gtgcacactg |
| 1261 | gggagcggcc ccacaagtgc tcggtgtgcc acaagcgctt |
| | cagcagctcc agtaacctca |
| 1321 | agacccacct gcgcctgcac tccggggccc ggcccttcca |
| | gtgcagtgtc tgccggagtc |
| 1381 | gcttcaccca gcacatccac ctgaagctgc accatcggct |
| | gcatgcccca cagccctgtg |
| 1441 | gcctggtgca cacccagctg cccctggcct ctctggcctg |
| | ccttgcccaa tggcaccagg |
| 1501 | gggcactaga tcttatggcg gtggcatctg agaaacacat |
| | gggctatgac atagatgagg |
| 1561 | tcaaagtgtc ctcgacatcc caggggaaag caagagcagt |
| | gagcctgagc agtgccggga |
| 1621 | ctcccctggt gatggggcag gaccagaaca attaaaaatg |
| | tttcttctgt caaaaaaaaa |
| 1681 | aaaaaaa |

Thymocyte Selection-Associated High Mobility Group Box Protein (TOX)

TOX, a nuclear factor, was recently identified as a crucial regulator of the differentiation of tumor-specific T (TST) cells (Scott et al., (2019), Nature 571, 270-274). TOXis highly expressed in dysfunctional TST cells from tumors and in exhausted T cells during chronic viral infection. Expression of TOXis driven by chronic T cell receptor stimulation and activation of nuclear factor of activated T-cells (NFAT). Ectopic expression of TOX in effector T cells in vitro induced a transcriptional program associated with T cell exhaustion.

An exemplary TOX amino acid sequence is provided at NCBI Accession No. NP_055544, version NP_055544.1, incorporated herein by reference and set forth below (SEQ ID NO: 5):

| | |
|---|---|
| 1 | mkeesaaqlg cchrpmalgg tggslspsld fqlfrgdqvf |
| | sacrplpdmv dahgpscasw |
| 61 | lcplplapgr sallaclqdl dlnlctpqpa plgtdlqglq |
| | edalsmkhep pglqasstdd |
| 121 | kkftvkypqn kdklgkqper agegapcpaf sshnsssppp |
| | lqnrkspspl afcpcppvns |
| 181 | iskelpfllh afypgyplll ppphlftyga lpsdqcphll |
| | mlpqdpsypt mampsllmmv |
| 241 | nelghpsarw etllpypgaf qasgqalpsq arnpgagaap |
| | tdspglergg maspakrvpl |
| 301 | ssqtgtaalp yplkkkngki lyecnicgks fgqlsnlkvh |
| | lrvhsgerpf qcalcqksft |
| 361 | qlahlqkhhl vhtgerphkc svchkrfsss snlkthlrlh |
| | sgarpfqcsv crsrftqhih |
| 421 | lklhhrlhap qpcglvhtql plaslaclaq whqgaldlma |
| | vasekhmgyd idevkvssts |
| 481 | qgkaravsls sagtplvmgq dqnn |

An exemplary TOX nucleic acid sequence is provided at NCBI Accession No. NM_014729, version NM_014729.3, incorporated herein by reference and set forth below (SEQ ID NO: 6):

| | |
|---|---|
| 1 | ctcttcttct taaacaaacc acaaacggat gtgagggaag |
| | gaaggtgttt cttttactcc |
| 61 | tgagcccaga cacctcactc tgttccgtct aagcttgttt |
| | tgctgaacac ttttttttaa |
| 121 | aaaaggaaaa agaaaaggag ttgcttgatg tgagagtgaa |
| | atggacgtaa gattttatcc |
| 181 | acctccagcc cagcccgccg ctgcgcccga cgctccctgt |
| | ctgggacctt ctccctgcct |
| 241 | ggacccctac tattgcaaca agtttgacgg tgagaacatg |
| | tatatgagca tgacagagcc |
| 301 | gagccaggac tatgtgccag ccagccagtc ctaccctggt |
| | ccaagcctgg aaagtgaaga |
| 361 | cttcaacatt ccaccaatta ctcctccttc cctcccagac |
| | cactcgctgg tgcacctgaa |
| 421 | tgaagttgag tctggttacc attctctgtg tcaccccatg |
| | aaccataatg gcctgctacc |
| 481 | atttcatcca caaaacatgg acctccctga aatcacagtc |
| | tccaatatgc tgggccagga |
| 541 | tggaacactg ctttctaatt ccatttctgt gatgccagat |
| | atacgaaacc cagaaggaac |
| 601 | tcagtacagt tcccatcctc agatggcagc catgagacca |
| | aggggccagc ctgcagacat |
| 661 | caggcagcag ccaggaatga tgccacatgg ccagctgact |
| | accattaacc agtcacagct |
| 721 | aagtgctcaa cttggtttga atatgggagg aagcaatgtt |
| | ccccacaact caccatctcc |
| 781 | acctggaagc aagtctgcaa ctccttcacc atccagttca |
| | gtgcatgaag atgaaggcga |
| 841 | tgatacctct aagatcaatg gtggagagaa gcggcctgcc |
| | tctgatatgg ggaaaaaacc |
| 901 | aaaaactccc aaaaagaaga agaagaagga tcccaatgag |
| | ccccagaagc ctgtgtctgc |
| 961 | ctatgcgtta ttctttcgtg atactcaggc cgccatcaag |
| | ggccaaaatc caaacgctac |
| 1021 | ctttggcgaa gtctctaaaa ttgtggcttc aatgtgggac |
| | ggtttaggag aagagcaaaa |
| 1081 | acaggtctat aaaaagaaaa ccgaggctgc gaagaaggag |
| | tacctgaagc aactcgcagc |
| 1141 | atacagagcc agccttgtat ccaagagcta cagtgaacct |
| | gttgacgtga agacatctca |
| 1201 | acctcctcag ctgatcaatt cgaagccgtc ggtgttccat |
| | gggcccagcc aggcccactc |
| 1261 | ggccctgtac ctaagttccc actatcacca acaaccggga |
| | atgaatcctc acctaactgc |
| 1321 | catgcatcct agtctcccca ggaacatagc ccccaagccg |
| | aataaccaaa tgccagtgac |
| 1381 | tgtctctata gcaaacatgg ctgtgtcccc tcctcctccc |
| | ctccagatca gcccgcctct |
| 1441 | tcaccagcat ctcaacatgc agcagcacca gccgctcacc |
| | atgcagcagc cccttgggaa |
| 1501 | ccagctcccc atgcaggtcc agtctgcctt acactcaccc |
| | accatgcagc aaggatttac |
| 1561 | tcttcaaccc gactatcaga ctattatcaa tcctacatct |
| | acagctgcac aagttgtcac |
| 1621 | ccaggcaatg gagtatgtgc gttcggggtg cagaaatcct |
| | cccccacaac cggtggactg |
| 1681 | gaataacgac tactgcagta gtgggggcat gcagagggac |
| | aaagcactgt accttacttg |
| 1741 | agaatctgaa cacctcttct ttccactgag gaattcaggg |
| | aagtgttttc accatggatt |
| 1801 | gctttgtaca gtcaaggcag ttctccattt tattagaaaa |
| | tacaagttgc taagcactta |
| 1861 | ggaccatttg agcttgtggg tcacccactc tggaagaaat |
| | agtcatgctt ctttattatt |
| 1921 | tttttaatcc tttatggaca ttgtttttct tcttccctga |
| | aggaaatttg gaccattcag |
| 1981 | attttatgtt ggttttttgc tgtgaagtgc tgcgctctag |
| | taactgcctt agcaactgta |
| 2041 | gatgtctcgg ataaaagtcc tggattttcc attggttttc |
| | ataatgggtg tttatatgaa |
| 2101 | actactaaag actttttaaa tggcttgatg tagcagtcat |
| | agcaagtttg taaatagcat |
| 2161 | ctatgttaca ctctcctaga gtataaaatg tgaatgtttt |
| | tgtagctaaa ttgtaattga |
| 2221 | aactggctca ttccagttta ttgatttcac aataggggtt |
| | aaattggcaa acattcatat |
| 2281 | ttttacttca tttttaaaac aactgactga tagttctata |
| | ttttcaaaat atttgaaaat |
| 2341 | aaaaagtatt cccaagtgat tttaatttaa aaacaaattg |
| | gctttgtctc attgatcaga |
| 2401 | caaaaagaaa ctagtattaa gggaagcgca aacacattta |
| | ttttgtactg cagaaaaatt |
| 2461 | gcttttttgt atcacttttt gtgtaatggt tagtaaatgt |
| | catttaagtc cttttatgta |
| 2521 | taaaactgcc aaatgcttac ctggtatttt attagatgca |
| | gaaacagatt ggaaacagct |
| 2581 | aaattacaac ttttacatat ggctctgtct tattgtttct |
| | tcatactgtg tctgtattta |
| 2641 | atcttttttt atggaacctg ttgcgcctat ttatgaaata |
| | ataaatatag gtgtttgtaa |
| 2701 | gtaaatttgt tagtatttga aagaggtttc tttgatgttt |
| | taacttttgc tggcaaaaaa |
| 2761 | aaattcacgc ttggtgtgaa tactttatta tttagttttt |
| | acagtaacat gaataaagcc |
| 2821 | aaacctgctt ttcatttagc agcaaattaa agtaaccagt |
| | ccttatttct gcatttcttt |
| 2881 | ggttgatgca aacaaaaaac tattatattt aagaacttta |
| | tttcttcata cgacataaca |
| 2941 | gaattgccct ccaagtcaca caagctccaa gactaaacaa |
| | acagacaggt cctctgtctt |
| 3001 | aaaaaggtta cttcttggtt ctcagctggt tctagtcaat |
| | tctgaaccac caccccccgc |
| 3061 | cccccgcaaa aaagtaaaag tcaaaccaaa cttcctcaag |
| | ctgcatgctt ttcacaaaat |
| 3121 | ccagaaagca tttaagaatt gaactagggg ctggaagaag |
| | tgaaagggaa gcatctaaaa |
| 3181 | atgaaaggtg agtaaccaga tagcaaaaga aaagggaaag |
| | ccatccaaat ttgaaagctg |
| 3241 | ttgatagaaa ttgagattct tgctgtcttt tgtgcctcta |
| | caagctacta ctcattccag |
| 3301 | aattcctggg tcttccaaga ggattcttaa ggtaccagag |
| | atttgctagg gaaccaaaag |
| 3361 | tgcttgagaa tctgcctgag ggcttgcata gctttcacat |
| | taaaaaaaga aaaagctagc |
| 3421 | agatttactc ctttttaggg gatcatatca agaaagttag |
| | tctggttgga aaccaagaga |
| 3481 | atggctgatg tctctttctt ggaatatgtg aaataaattt |
| | agcagtttaa ctaaatacaa |
| 3541 | atatatgcat tgtgtaatcc actcagaatt aaacagacaa |
| | aaggtatgct tgctttggaa |
| 3601 | tgattttagg cattgtacaa ccttgaatca cttgagcatg |
| | taataactaa taaataatgc |
| 3661 | agatccatgt gattattaaa atgactgtag ctgagagctc |
| | taattttcct gtcttgaaac |
| 3721 | tgtataagaa ctcatgtgat taagttcaca gtttattgtt |
| | tgtctgttta gtattttaga |
| 3781 | aatataccag cactactaat taactaatgt cttttattta |
| | ttatattatg ataaagtaaa |
| 3841 | aatttcactt gcattaagtc taaactgaga aggtaattac |
| | tgggaggaga atgagcagct |
| 3901 | ttgactttga caggcggttt gtgcaggaaa gcacagtgcc |
| | gtgttgttta cagcttttct |
| 3961 | agagcagctg tgcgaccagg gtagagagtg ttgaaattca |
| | ataccaaata cagtaaaaac |
| 4021 | aaatgtaaat aaaagaaaac acatcatcaa taaaactgtt |
| | attatgcgtg accgta |

As illustrated in the Examples that follow, to systematically discover the cellular determinants of response to PD-1 blockade, an in-depth analysis of single-cell transcriptome data generated from serially collected bone marrow samples from 6 RT patients treated with PD-1 blockade was performed. RT, a hematologic malignancy, provides a unique model for the examination of the immune populations associated with tumor response within the context of the microenvironment of an immune organ. Immunohistochemical studies were included in the analysis.

The study identified distinct cell populations, already detectable at baseline, associated with therapeutic response (i.e., clonally expanded CD8 effector/effector memory cells marked by the transcription factor ZNF683 and cytotoxic CD4 T cells) and lack of response (increased exhaustion). This result was confirmed in an independent cohort of RT patients. Markers that can serve to identify patients (prior to treatment) who may benefit from PD-1 blockade were identified.

World Health Organization Criteria

The WHO Criteria for evaluating the effectiveness of anti-cancer agents on tumor shrinkage, developed in the 1970s by the International Union Against Cancer and the World Health Organization, represented the first generally agreed specific criteria for the codification of tumor response evaluation. These criteria were first published in 1981 (Miller et al., 1981 Clin Cancer Res., 47(1): 207-14, incorporated herein by reference). WHO Criteria proposed>50% tumor shrinkage for a Partial Response and >25% tumor increase for Progressive Disease.

### Response Evaluation Criteria in Solid Tumors (RECIST)

RECIST is a set of published rules that define when tumors in cancer patients improve (“respond”), stay the same (“stabilize”), or worsen (“progress”) during treatment (Eisenhauer et al., 2009 European Journal of Cancer, 45: 228-247, incorporated herein by reference). Only patients with measurable disease at baseline should be included in protocols where objective tumor response is the primary endpoint.

The response criteria for evaluation of target lesions are as follows:
Complete Response (CR): Disappearance of all target lesions.
Partial Response (PR): At least a 30% decrease in the sum of the longest diameter (LD) of target lesions, taking as reference the baseline sum LD.
Stable Disease (SD): Neither sufficient shrinkage to qualify for PR nor sufficient increase to qualify for PD, taking as reference the smallest sum LD since the treatment started.
Progressive Disease (PD): At least a 20% increase in the sum ofthe LD of target lesions, taking as reference the smallest sum LD recorded since the treatment started or the appearance of one or more new lesions.
   The response criteria for evaluation of non-target lesions are as follows:
Complete Response (CR): Disappearance of all non-target lesions and normalization of tumor marker level.
Incomplete Response/Stable Disease (SD): Persistence of one or more non-target lesion(s) or/and maintenance of tumor marker level above the normal limits.
Progressive Disease (PD): Appearance of one or more new lesions and/or unequivocal progression of existing non-target lesions.

The response criteria for evaluation of best overall response are as follows. The best overall response is the best response recorded from the start of the treatment until disease progression/recurrence (taking as reference for PD the smallest measurements recorded since the treatment started). In general, the patient's best response assignment will depend on the achievement of both measurement and confirmation criteria.
Patients with a global deterioration of health status requiring discontinuation of treatment without objective evidence of disease progression at that time should be classified as having “symptomatic deterioration”. Every effort should be made to document the objective progression even after discontinuation of treatment.
In some circumstances, it may be difficult to distinguish residual disease from normal tissue. When the evaluation of complete response depends on this determination, it is recommended that the residual lesion be investigated (fine needle aspirate/biopsy) to confirm the complete response status.

### Immune-Related Response Criteria

The immune-related response criteria (irRC) is a set of published rules that define when tumors in cancer patients improve (“respond”), stay the same (“stabilize”), or worsen (“progress”) during treatment, where the compound being evaluated is an immuno-oncology drug. The Immune-Related Response Criteria, first published in 2009 (Wolchok et al., 2009 Clin Cancer Res, 15(23):7412, incorporated herein by reference), arose out of observations that immuno-oncology drugs would fail in clinical trials that measured responses using the WHO or RECIST Criteria. These criteria could not account for the time gap in many patients between initial treatment and the apparent action of the immune system to reduce the tumor burden. The key driver in the development of the irRC was the observation that, in studies of various cancer therapies derived from the immune system such as cytokines and monoclonal antibodies, the looked-for Complete and Partial Responses as well as Stable Disease only occurred after an increase in tumor burden that the conventional RECIST Criteria would have dubbed “Progressive Disease’. RECIST failed to take account of the delay between dosing and an observed anti-tumor T cell response, so that otherwise ‘successful’ drugs—that is, drugs which ultimately prolonged life—failed in clinical trials.

The irRC are based on the WHO Criteria; however, the measurement of tumor burden and the assessment of immune-related response have been modified as set forth below.

### Measurement of Tumor Burden

In the irRC, tumor burden is measured by combining ‘index’ lesions with new lesions. Ordinarily, tumor burden would be measured with a limited number of ‘index’ lesions (that is, the largest identifiable lesions) at baseline, with new lesions identified at subsequent timepoints counting as ‘Progressive Disease’. In the irRC, by contrast, new lesions are a change in tumor burden. The irRC retained the bidirectional measurement of lesions that had originally been laid down in the WHO Criteria.

### Assessment of Immune-Related Response

In the irRC, an immune-related Complete Response (irCR) is the disappearance of all lesions, measured or unmeasured, and no new lesions; an immune-related Partial Response (irPR) is a 50% drop in tumor burden from baseline as defined by the irRC; and immune-related Progressive Disease (irPD) is a 25% increase in tumor burden from the lowest level recorded. Everything else is considered immune-related Stable Disease (irSD). Even if tumor burden is rising, the immune system is likely to “kick in” some months after first dosing and lead to an eventual decline in tumor burden for many patients. The 25% threshold accounts for this apparent delay.

### Gene Expression Profiling

In general, methods of gene expression profiling may be divided into two large groups: methods based on hybridization analysis of polynucleotides and methods based on sequencing of polynucleotides. Methods known in the art for the quantification of mRNA expression in a sample include northern blotting and in situhybridization, RNAse protection assays, RNA-seq, and reverse transcription polymerase chain reaction (RT-PCR). Alternatively, antibodies that recognize specific duplexes are employed, including DNA duplexes, RNA duplexes, and DNA-RNA hybrid duplexes or DNA-protein duplexes. Representative methods for sequencing-based gene expression analysis include Serial Analysis of Gene Expression (SAGE) and gene expression analysis by massively parallel signature sequencing (MPSS). For example, RT-PCR is used to compare mRNA levels in different sample populations, in normal and tumor tissues, with or without drug treatment, to characterize patterns of gene expression, to discriminate between closely related mRNAs, and/or to analyze RNA structure.

In some cases, a first step in gene expression profiling by RT-PCR is the reverse transcription of the RNA template into cDNA. This is followed by amplification in a PCR reaction. For example, extracted RNA is reverse-transcribed using a GeneAmp® RNA PCR kit (Perkin Elmer, Calif., USA), following the manufacturer's instructions. The cDNA is then used as template in a subsequent PCR amplification and quantitative analysis using, for example, a TaqMan™ RTM (Life Technologies™, Inc., Grand Island, N.Y.) assay.

### Microarrays

Differential gene expression can also be identified or confirmed using a microarray technique. In these methods, polynucleotide sequences of interest (including cDNAs and oligonucleotides) are plated, or arrayed, on a microchip substrate. The arrayed sequences are then hybridized with specific DNA probes from cells or tissues of interest. Just as in the RT-PCR method, the source of mRNA typically is total RNA isolated from human tumors or tumor cell lines and corresponding normal tissues or cell lines. Thus, RNA is isolated from a variety of primary tumors or tumor cell lines. If the source of mRNA is a primary tumor, mRNA is extracted from frozen or archived tissue samples.

In the microarray technique, PCR-amplified inserts of cDNA clones are applied to a substrate in a dense array. The microarrayed genes, immobilized on the microchip, are suitable for hybridization under stringent conditions.

In some cases, fluorescently labeled cDNA probes are generated through incorporation of fluorescent nucleotides by reverse transcription of RNA extracted from tissues of interest (e.g., leukemia tissue). Labeled cDNA probes applied to the chip hybridize with specificity to loci of DNA on the array. After washing to remove non-specifically bound probes, the chip is scanned by confocal laser microscopy or by another detection method, such as a charge-coupled device (CCD) camera. Quantification of hybridization of each arrayed element allows for assessment of corresponding mRNA abundance.

In some configurations, dual color fluorescence is used. With dual color fluorescence, separately labeled cDNA probes generated from two sources of RNA are hybridized pairwise to the array. The relative abundance of the transcripts from the two sources corresponding to each specified gene is thus determined simultaneously. In various configurations, the miniaturized scale of the hybridization can afford a convenient and rapid evaluation of the expression pattern for large numbers of genes. In various configurations, such methods can have sensitivity required to detect rare transcripts, which are expressed at fewer than 1000, fewer than 100, or fewer than 10 copies per cell. In various configurations, such methods can detect at least approximately two-fold differences in expression levels (Schena et al., Proc. Natl. Acad. Sci. USA 93(2): 106-149 (1996)). In various configurations, microarray analysis is performed by commercially available equipment, following manufacturer's protocols, such as by using the Affymetrix™ GenChip technology, or Incyte's microarray technology.

### RNA-Seq

RNA sequencing (RNA-seq), also called whole transcriptome shotgun sequencing (WTSS), uses next-generation sequencing (NGS) to reveal the presence and quantity of RNA in a biological sample at a given moment in time.

RNA-Seq is used to analyze the continually changing cellular transcriptome. See, e.g., Wang et al., 2009 Nat Rev Genet, 10(1): 57-63, incorporated herein by reference. Specifically, RNA-Seq facilitates the ability to look at alternative gene spliced transcripts, post-transcriptional modifications, gene fusion, mutations/SNPs and changes in gene expression. In addition to mRNA transcripts, RNA-Seq can look at different populations of RNA to include total RNA, small RNA, such as miRNA, tRNA, and ribosomal profiling. RNA-Seq can also be used to determine exon/intron boundaries and verify or amend previously annotated 5′ and 3′ gene boundaries.

Prior to RNA-Seq, gene expression studies were done with hybridization-based microarrays. Issues with microarrays include cross-hybridization artifacts, poor quantification of lowly and highly expressed genes, and needing to know the sequence of interest. Because of these technical issues, transcriptomics transitioned to sequencing-based methods. These progressed from Sanger sequencing of Expressed Sequence Tag libraries, to chemical tag-based methods (e.g., serial analysis of gene expression), and finally to the current technology, NGS of cDNA (notably RNA-Seq).

### Cellular Indexing of Transcriptomes and Epitopes by Sequencing (CITE-Seq)

Cellular indexing of transcriptomes and epitopes by sequencing (CITE-seq) is a single cell phenotyping method in which oligonucleotide-labeled antibodies are used to integrate cellular protein and transcriptome measurements into an efficient, single-cell sequenceable readout. CITE-seq is compatible with existing single-cell sequencing approaches and scales readily with throughput increases. The antibody-bound oligonucleotides act as synthetic transcripts that are captured during most large-scale oligodT-based scRNA-seq library preparation protocols (e.g., 10× Genomics, Drop-seq, ddSeq).

### Assay for Transposase-Accessible Chromatin Using Sequencing (ATAC-Seg)

ATAC-seq is a technique used in molecular biology to assess genome-wide chromatin accessibility. It identifies accessible DNA regions by probing open chromatin with hyperactive mutant Tn5 Transposase. The Tn5 Transposase then inserts sequencing adapters into open regions of the genome. While naturally occurring transposases have a low level of activity, ATAC-seq employs the mutated hyperactive transposase. In a process called “tagmentation”, Tn5 Transposase cleaves and tags double-stranded DNA with sequencing adaptors. The tagged DNA fragments are then purified, PCR-amplified, and sequenced using next-generation sequencing. Sequencing reads can then be used to infer regions of increased accessibility as well as to map regions of transcription factor binding sites and nucleosome positions. The number of reads for a region correlate with how open that chromatin is, at single nucleotide resolution. ATAC-seq is advantageous because it requires no sonication or phenol-chloroform extraction, no antibodies, no sensitive enzymatic, and can be completed in under three hours.

### Cleavage Under Targets and Tagmentation (CUT&Tag)

CUT&Tag is a molecular biology method used to investigate interactions between proteins and DNA and to identify DNA binding sites for their protein of interest. Unlike ChIP assays which requires crosslinked the cells or tissue, the starting material for is live permeabilized cells or isolated nuclei. In CUT&Tag protocols, cells are first permeabilized and incubated with an antibody immobilized on concanavalin A-coated magnetic beads. The use of beads facilitates the subsequent washing steps. Next, the cells are incubated with a primary antibody specific for the target protein of interest, followed by incubation with a secondary antibody. The cells are then incubated with assembled transposomes. The transposomes consist of protein A fused to the Tn5 transposase enzyme that is conjugated to next generation sequencing (NGS) adapters. After the incubation, unbound transposome is washed away using stringent conditions. Magnesium ions (Mg2+) are added to activate the reaction, which results in the chromatin being cut close to the protein binding site and the simultaneous addition of the NGS adapter DNA sequences. This method facilitates chromatin cleavage and library preparation in one single step. CUT&Tag is advantageous because the transposase only cuts chromatin at close proximity to the protein binding site, resulting in sequencing of shorter length DNA. This allows lower sequencing depth (3-5 million reads) to generate robust data, with lower background signal than most ChIP-Seq assays. Finally, because the CUT&Tag protocol uses intact cells as the starting material, it can be adapted to single-cell experiments (scCUT&Tag).

### Cyclic Immunofluorescence Imaging (CVCIF)

CyCIF is a robust and inexpensive method known in the art for highly multiplexed immunofluorescence imaging. The concept of repeatedly staining and imaging slides most commonly involves antibody stripping using denaturants. The method involves chemical inactivation of fluorophores after several rounds of immunofluorescence (Gerdes et al., 2013 Proc Natl Acad Sci USA., 110(29):11982-11987). The data collected using CyCIF allows investigation of complex associations and interdependencies between observed features and phenotypes.

CyCIF uses known and existing antibodies directly conjugated to fluorophores to construct images with up to 30 channels with sequential 4-6 channel imaging followed by fluorophore inactivation. Exemplary fluorophores that may be useful include Alexa Fluor® 488/555/647 fluorophores, which are substantially brighter and more photo-stable than antibodies conjugated to FITC/Cy3/Cy5 dyes (Panchuk-Voloshina et al., 1999 The Journal of Histochemistry and Cytochemistry: Official Journal of the Histochemistry Society, 47(9):1179-88.) Cell morphology is preserved through multiple rounds of CycIF, and unlike antibody-stripping methods, CycIF is optimized for imaging monolayers of cultured cells.

### Pharmaceutical Therapeutics

For therapeutic uses, the compositions or agents described herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplasia, i.e., the melanoma. Generally, amounts will be in the range of those used for other agents used in the treatment of other diseases associated with neoplasia, although in certain instances lower amounts will be needed because of the increased specificity of the compound. For example, a therapeutic compound is administered at a dosage that is cytotoxic to a neoplastic cell.

### Formulation of Pharmaceutical Compositions

The administration of a compound or a combination of compounds for the treatment of a neoplasia, e.g., a melanoma, may be by any suitable means that results in a concentration of the therapeutic that, combined with other components, is effective in ameliorating, reducing, or stabilizing a neoplasia. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Human dosage amounts effective for treating RT can initially be determined by extrapolating from the amount of compound used in mice, as a skilled artisan recognizes it is routine in the art to modify the dosage for humans compared to animal models. In certain embodiments it is envisioned that the dosage may vary from between about 1 sg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other cases, this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 mg/Kg body weight. In other aspects, it is envisaged that doses may be in the range of about 5 mg compound/Kg body to about 20 mg compound/Kg body. In other embodiments, the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

In some embodiments, the dosage amount may be volume dependent (e.g., mg/mL) in order to facilitate intravenous infusion. For example, the dosage amount of nivolumab may be 40 mg/4 mL, 100 mg/10 mL, and 240 mg/24 mL, with a recommended treatment dosage of 240 mgs administered intravenously every 2 weeks or 480 mgs administered intravenously every 4 weeks. In other embodiments, the dosage amount for pembrolizumab may be 100 mg/4 mL with a recommended treatment dosage of 200 mgs administered intravenously every 3 weeks. In other embodiments, the dosage formulation amount for cemiphimab may be 350 mg/7 mL with a recommended treatment dosage of 350 mgs administered intravenously every 3 weeks.

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target a neoplasia by using carriers or chemical derivatives to deliver the therapeutic agent to a particular cell type (e.g., neoplastic cell). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a neoplasia, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active antineoplastic therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Kits or Pharmaceutical Systems

The present compositions may be assembled into kits or pharmaceutical systems for use in ameliorating a neoplasia (e.g., melanoma). Kits or pharmaceutical systems according to this aspect of the invention comprise a carrier means, such as a box, carton, tube or the like, having in close confinement therein one or more container means, such as vials, tubes, ampoules, or bottles. The kits or pharmaceutical systems of the invention may also comprise associated instructions for using the agents of the invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Materials and Methods

The following materials and methods were utilized to generate the results described herein.

### Human Samples

Whole bone marrow and whole blood samples were obtained from RT and CLL patients enrolled on clinical trials of nivolumab plus ibrutinib therapy (NCT01328626, NCT02141282). Mononuclear bone marrow samples from control healthy donors were obtained through the tissue bank at Dana-Farber Cancer Institute (DFCI). They were approved by and conducted in accordance with the principles of the Declaration of Helsinki and with the approval of the Institutional Review Boards (IRB) of the University of Texas/MD Anderson Cancer Center (MDAC) or of Dana-Farber Cancer Institute. From clinical trial patients, blood, marrow and/or tissue tumor samples were collected at baseline, response assessment, and at relapse. Samples from MDAC were obtained in heparin green top tubes and placed on ice after collection. They were mixed at a 1:1 ratio with Freezing media (80% FCS and 20% DMSO) and cryopreserved and stored in liquid nitrogen until analysis. Healthy controls from DFCI underwent mononuclear cell isolation by Ficoll gradient prior to being preserved in freezing media (fetal calf serum (FCS) with final concentration 10% DMSO) and cryopreserved using similar methods.

### PBMCs for Flow Cytometry

Peripheral blood mononuclear cells were obtained from healthy donors and isolated using Ficoll gradient centrifugation. Cells were cryopreserved using FCS with 10% DMSO.

### Hobit Antibody for Flow Cytometry

BD anti-ZNF683 IgM (clone Sanquin-Hobit/1) was purchased as a custom order (BD Pharmigen). APC conjugation was performed using AbCam Lightening-Link (ab201807).

### Hobit Antibody (IgG) for IP

Anti-znf684 hybridoma cell line (PCRP-ZNF683-1E4) was purchased and grown. 5-6 micrograms of purified antibody was obtained.

### Flow Cytometrv Antibodies for Validation

CD3 BV421(300434), CD4 BV605 (300556), CD8 BV785 (301046), CD45RA FITC (304106), CD62L PE Cy7 (304822), CD127 BV650 (351325), PD-1 APC Cy7(329922), GZMB -AF700 (372222) from Biolegend and Anti-TOX PE (Miltenyi 130-107-784 1).

### Flow Cytometry Validation

PBMCs and clinical trial samples were thawed in PBS with 10% FCS and 10% DNase. Samples were then spun for 10 minutes at 300×g. Cells were resuspended in RPMI with 10% FCS and 10% DNase (StemCell technologies) and incubated at 4 hours at 37 degrees Celsius prior to flow cytometry staining. Single color controls and FMO control was used for Hobit staining.

After 4 hours, cells were washed with PBS and stained with zombie aqua viability marker (Biolegend, 423101 & 423102). Cells were then incubated with FcBlock (Biolegend Trustain FcX; 422301 & 422302) followed by surface staining. Cells were then washed and fixed using BD transcription factor staining kit (BD Pharmingen, 562574) and permeabilized overnight prior to intracellular staining per manufacturer protocol. Cells were washed and flow cytometry was performed using a BD™ LSR Fortessa™ 4 laser instrument. Data analysis was performed using Flow Jo.

### Flow Cvtometry Sorting

Cells were thawed by drop-wise addition of warmed media (RPMI 10% FCS 1% P/S) and stained with antibodies (Biolegend CD5 FITC 364022, CD19 PE-Cy7 302216, CD3 PB 300330) and 7-AAD (Biolegend 420404) before being resuspended in PBS-0.04% BSA (NEB/Invitrogen).

Viable CD5+ CD19+ population was sorted for chronic lymphocytic leukemia (CLL) and viable CD5−CD19− population for immune fraction. In marrow samples where Richter's transformation (RT) tumor was present, RT and CLL fractions were sorted by size based on the increased forward scatter (FSC) of RT cells.

Single-Cell RNA-Sequencing (scRNA-Sea)

For the discovery cohort, scRNA-sequencing was conducted on 5,000 to 10,000 single-cells using the 10× genomics 3′ version 2 reagent kit and run on a chromium controller according to manufacturer's instructions. Libraries were pooled and sequenced on MiSeq or NovaSeq (Illumina®). For single cell sequencing with T cell receptors (TCR), the 10× genomics 5′ version 2 library kit was used with human T cell V(D)J enrichment kit.

### (CITE-Seq) and TCR

For CITE-seq, a similar sorting strategy was used. Bone marrow samples were thawed into warmed phosphate-buffered saline (PBS)/10% FCS and washed with PBS. Cells were stained with Zombie Violet™ viability marker (BioLegend®) followed by CD19 (BioLegend®) and glycophorin (BD™, CD235 antibody) to mark tumor B cells and erythrocyte precursors respectively. The non-stained population (CD19-glycophorinA-) population was sorted for sc-RNA sequencing. Cells were then pelleted and stained with 55 antibodies (supp methods table) from BioLegend® Total-Seq™-C system according to manufacturer protocol in PBS-0.04% bovine serum albumin (BSA). After washing, the cells were sequenced using the 5′ V2 kit with gene/protein (10×).

### Nucleofection

### T Cells

Healthy donor T cells were isolated from peripheral blood of healthy donors as follows. Healthy donor blood (Kraft Donor Center) was obtained and ficoll gradient centrifugation was performed to isolate mononuclear cells. Subsequent T cell isolation was performed using bead-based magnestic separation (Biolegend Mojosort CD3 selection kit). Five (5) million isolated T cells were then nucleofected using EO-115 program and Lonza P3 primary cell kit on Lonza Core Unit X. Media was changed approximately 12 hours post-nucelofection with addition of cytokines (50 U/mL IL-7 and IL-15) and CD3/28 bead stimulation (Dynabeads). Cells were cultured with beads for 6 days prior to bead removal wit new media added as needed. Cells were then expanded post-beads in the presence of puromycin (0.250 μg/mL) for 3-5 days. Percent GFP positivity was then assessed using flow cytometry. Cells were then washed and plated with or without 1 microgram/mL of doxycycline for 48 hours prior to experimental endpoints.

### Jurkat Cells

Jurkat cells, clone E6-1), were purchased fresh from ATCC (TIB-152) and grown in culture with RPMI 10% FCS. One (1) million cells of early passage Jurkats were nucleofected with 15 μg of Hobit-FLAG plasmid or 15 μg of control plasmid (pSBtet-GP) with 15 μg of pCMV(CAT)T7-SB100. Nucleofection was performed using SE kit (Lonza) per manufacturer protocol for E6-1 using the Core Unit X (Lonza). Media was changed the following day and cells were rested in culture for 3 days prior to 5-7 days of puromycin selection (0.250 μg/mL). After one week, high nucleofection efficiency was confirmed by flow cytometry for GFP. Cells were then washed and plated with or without 1 microgram/mL doxycycline for 48 hours prior to experimental endpoints.

### Western Blot

Hobit protein expression was confirmed using Western Blot. Three to five (3-5) million cells were pelleted and nuclear isolates were obtained (Pierce NE-PER Nuclear Extract Kit) per manufacturer protocol. Protein was stained and loaded onto a NuPage 4-12% Bis-Tris gel and run in NuPAGE Mops Sds Running Buffer (BioRad). Transfer was performed using iBlot 2 gel transfer system and membrane was blocked using 5% BSA in TBS-T. Primary antibody staining was performed with anti-ZNF683 (Thermo Fisher, PA5-41242) or anti-FLAG (Cell Signaling Technology, 2368S) with histone H3 (CST, 9715S) loading control followed by anti-rabbit or anti-mouse HRP secondary antibody (7074S CST for anti-rabbit, NA931V for anti-mouse (GE)). Membranes were developed using Pierce Supersignal Pico kit and captured using BioRad Chemidoc.

Cleavage under Targets and Tagmentation (CUT&Tag)

The CUT&Tag was performed on one hundred thousand (100,000) Jurkats or CD8 T cells using published protocol (protocols.io V2) with modification of using CUTANA® pAG-Tn5 for ChIC/CUT&Tag (EpiCypher). Anti-znf684 hybridoma cell line (PCRP-ZNF683-1E4) was purchased and grown per recommendations by the DFCI Monoclonal Antibody Core Facility. Five to six (5-6) micrograms of purified antibody was obtained. Primary antibodies used were anti-FLAG (CST 2368S), H3K4Me2(Upstate 07-030), H3K27Me3 (CST C36B11), anti-Hobit (PCRP-ZNF683-1E4 purified), and IgG isotype control. Secondary antibodies were Guinea Pig Anti-rabbit (Abcam, ABIN101961) and Rabbit-anti-mouse (Abcam, AB6709). Libraries were visualized prior to sequencing on a Agilent Bioanalyzer with high-sensitivity DNA kit.

### Data Analysis

Data Processing of scRNA-Seq Libraries

ScRNA-seq reads were processed, aligned to the Human Genome version 19 (Hg19) reference genome and filtered using the Cell Ranger pipeline (v2.0.0, 10× Genomics). Filtered feature-barcode matrices containing detected cellular barcodes were used in further analysis. Initially, each sample was processed individually with Pagoda2. This included quality control and count normalization. Cells were filtered based on size with a minimum cell size of 500 Unique molecular identifiers (UMIs), maximum of 8,000 UMIs (12,000 for RT samples due to larger cell size of RT cells), and >10 percentage of mitochondrial genes and low expressed genes were removed if detected in 10 cells or less. Across all samples, a total of 196,191 cells passed these filters and were subject to further analysis of separate tumor and immune compartments.

### Data Processing of CITE-Seq and TCR Libraries

CITE-seq and TCR reads were aligned to the Genome Reference Consortium Human Build 38 (GRCh38) reference genome and transcripts quantified using the Cell Ranger pipeline (v3.0.2, 10× Genomics). Further processing of gene expression data was as described above. Protein expression data was processed per sample and normalized by the summed count of isotypes (IgG1, IgG2a, IgG2b antibodies). Total number of cells captured with linked gene, protein and TCR data is—with an average—genes/cell and—protein/cell.

### Joint Clustering

In order to compare cell proportions and states across multiple time points and samples, data was combined using Conos. Conos maps cell types across a sample panel through pairwise comparisons between each pair in the panel, thereby generating a global joint graph. Recurrent cell populations tend to map to each other, thus forming community-like structures within the joint graph. Inter-sample pairs are mapped using nearest-neighbor graphs in a reduced expression space, common principal component analysis (CPCA) was used herein. Joint clusters were subsequently detected using the Leiden community detection method.

For analysis of the immune cells, a total of 20 samples, which resulted in 13 transcriptionally distinct clusters, was assembled using the default resolution parameter (1.0). Lymphocyte clusters were identified by marker genes and cells from these were extracted and subclustered. Here, the resolution was increased to 1.5 and detected 11 distinct clusters. Marker genes for each cluster were computed and these were used along with expression of canonical markers to annotate clusters. Differential gene expression between responders and non-responders was performed using DESeq2. As a cell single cell might not be a truly independent observation, count data was concatenated by summation within a cluster per sample, thus generating a bulk RNA sequencing like count matrix. This naturally generated higher counts indicating a greater confidence that a given gene was observed. DESeq2 has its own normalization step that accounts for differences in library size, and therefore raw count data was used as input for all DESeq2 runs. This normalization accounts for instances with uneven sample contribution to a cluster.

Concurrently, a joint analysis of tumor samples was performed. Twelve distinct clusters were detected, including four immune cells (spillover from sorting), using a resolution of 1.0. Only clusters consisting of malignant B cells were considered in subsequent analysis. Differential gene expression between RT and CLL cells was performed using DESeq2 with the same approach as described earlier. Gene set enrichment analysis (GSEA) was run on differentially expressed genes list using the C1 and C2 gene sets from the molecular signature database (MSigDb).

### Addition of Normals and CITE-Seq Samples

Lymphocytes from additional samples, including 28 normal marrows and 4 CITE-seq samples, were included in our lymphocyte subclustering by label propagation. This is done by computing the probabilities for each cell belonging to each of the 11 clusters.

Cluster annotations were confirmed using the normalized protein-linked expression from CITE-seq samples.

### TCR Repertoires

Cell barcodes with corresponding alpha and beta chain nucleotides sequences were extracted and only cells with productive TCRs were considered. Productive TCRs are defined as having either one alpha chain and one beta chain, two alpha chains and one beta chain, or one alpha chain and two beta chains. Of all TCRs—% and—% were defined as productive for RT R2 and RT NR1, respectively. For the responder and non-responder, unique clonotype frequencies were summarized and evaluated pre and post therapy.

### TCR Cloning and Functional Testing

### TCR Cloning

T cell beta chain VDJ sequences and alpha AJ sequences were obtained from 10× sequencing. Gene block constructs were synthesized using paired alpha and beta sequences with mouse T cell receptor beta constant (TRBC) and T cell receptor alpha constant (TRAC) linked by P2A for the top 10 clonotypes of RT R2 (IDT® DNA Technologies). Gene blocks were cloned into a PEW-pEFlalpha backbone using Gibson assembly (New England Biolabs®) electrocompetent transformation protocol with 5-alpha competent E. coli cells (New England Biolabs®). Colonies were selected and screened for insert by sequencing before undergoing plasmid DNA extraction (Endotoxin-Free Plasmid Maxiprep, Qiagen).

### Virus: Lentivirus Production and Purification

To produce lentivirus, 2 million HEK293T cells were seeded per well in a 10 cm plate in 2.7 mL of antibiotic-free Dulbecco's Modified Eagle Medium (DMEM) supplemented with 10% FBS. For each well, 150 μL of OptiMEM™ (Life Technologies™) was mixed with 5 μg of pLKO5_sgRNA plasmid, 0.4 μg of pVSV.G, and 1.5 μg of psPAX2 (Addgene #12260). Separately, 9 μL of Lipofectamine® 2000 (Life Technologies™) was diluted in 150 μL OptiMEM™. After a 15 min incubation at room temperature, the DNA and Lipofectamine® mixes were combined and incubated together at room temperature for 30 min before being added to the cells. After 12 h, the media was changed to DMEM supplemented with 20% FBS. 48 h post-transfection, 3 mL of media was removed and filtered through a 0.45 μm low protein binding membrane (Millipore™ Steriflip™ HV/PVDF) and added to 1 mL of Lenti-X™ Concentrator (Clontech). This mixture was then incubated at 4° C. for 2 h, and centrifuged at 1500×g for 45 min at 4° C. The pellet was resuspended in 100 μL of PBS and stored in aliquots at −80° C.

### Hobit Plasmid Generation

pSBtet-GP was digested with ClaI and SfiI enzymes (NEB) and gel purified (Wizard SV gel purification kit, Promega). A hobit gene block of active isoform was obtained from IDT DNA. Hobit gene was PCR amplified with FLAG-tag addition, gel purified and cloned into digested pSBtet-GP by Gibson cloning (NEB Gibson). Two (2) μL of Gibson reaction was mixed with Z-comp competent E coli cells and plated onto ampicillin plates. Colonies were selected and screened by sequencing the following day. Maxipreps were then performed of correct plasmid (Hobit-FLAG)

### Whole-Exome Sequencing and Data Analyses

Library construction from RT, CLL and matched germline (T cell) DNA of Patients RT R2, RT R3, and RT R4 was performed as previously described (Gruber et al., 2019 Nature, 570(7762):474-479). Alignments to hg19 using bwa version 0.5.9-r1649 and quality control were performed using the Picard (http://picard.sourceforge.net/) and Firehose (pipelines at the Broad Institute). Firehose is a framework combining workflows for the analysis of cancer-sequencing data. The workflows perform quality control, local re-alignment, mutation calling, small insertion and deletion identification, rearrangement detection and coverage calculations, among other analyses.

### Identification of Somatic Mutations

Sequencing output was processed with the Picard and GenomeAnalysisToolkit (GATK) toolkits developed at the Broad Institute, a process that involves marking duplicate reads, recalibrating base qualities and realigning around somatic small insertions and deletions (sINDELs). All BAM files were generated by aligning with bwa version 0.5.9 to the NCBI Human Reference Genome Build hg19. Prior to variant calling, the impact of oxidative damage (oxoG) and formalin-fixed paraffin-embedded (FFPE) damage to DNA during sequencing was quantified according to (Costello et al). The cross-sample contamination was measured with ContEst based on the allele fraction of homozygous single nucleotide polymorphisms (SNPs), and this measurement was used in MuTect. From the aligned BAM files, somatic alterations were identified using a set of tools developed at the Broad Institute (www.broadinstitute.org/cancer/cga). The details of our sequencing data processing have been described elsewhere.

Following standard procedure, synonymous single nucleotide variants (sSNVs) were detected using MuTect9 (version 1.1.6); somatic insertions and deletions (sINDELs) were detected using Strelka. A stringent set of filters was then applied to improve the specificity of our sSNV and sINDEL calls and remove likely FFPE artifacts. An allele fraction specific panel-of-normals filter, which compares the detected variants to a large panel ofnormal exomes and removes variants that were observed in the panel-of-normals, was applied. Then a realignment based filter, which removes variants that can be attributed entirely to ambiguously mapped reads, was applied. All filtered events in candidate CLL genes were also manually reviewed using the Integrated Genomics Viewer (IGV). In the matched sample sets from6 individuals, “forced calling” was used to quantify the number of reads supporting the altemate and reference alleles at sites which were detected in any sample from that individual. Estimation of and correction for tumor contamination in normal was performed using the deTiN algorithm to recover somatic mutations that would have otherwise been filtered out due to evidence of the mutation in the normal. To address the lack of a matched normal sample (e.g., in Patient 3 and Patient 6), a stringent panel-of-normals and population allele frequency criteria were used and non-coding variants were excluded from analysis. Furthermore, parental OCI-Ly1-S cells were used as a source control DNA in order to highlight sSNVs that were acquired in the resistant OCI-Ly1-R cells. Reference lists for sSNVs and sINDELs in known putative CLL driver genes as well as for recurrent CNAs were concatenated based on previous sequencing studies of large CLL cohorts. Total copy number was measured using ReCapSeg (broadinstitute.org/cancer/cga), then segmented into allelic copy number with AllelicCapSeg based on heterozygous germline sites detected with HaplotypeCaller according to the protocol described previously (archive.broadinstitute.org/cancer/cga/acsbeta).

Estimation of Mutation Cancer Cell Fraction Using ABSOLUTE and Clonal Evolution Mapping

The cancer cell fraction (CCF) (represented as a probability density distribution E [0, 1]) of individual somatic alterations were estimated using the ABSOLUTE algorithm (v1.5) which calculates the sample purity, ploidy, and local absolute DNA copy-number of each mutation, as previously described. CCFs were clustered as previously described to delineate distinct subclonal populations. Phylogenetic relationships between these populations were inferred using patterns of shared mutations and CCF using the PhylogicNDT analysis.

### RNA Sequencing

RNA was isolated from 2 million peripheral blood cells (Qiagen RNeasy) and bulk TCR sequencing was performed using a MiSeq or Novoseq System (Illumina®). See, Keskin et al., 2019 Nature, 565(7739): 234-239, incorporated herein by reference.

### Assay for Transposase-Accessible Chromatin Using Sequencing (ATAC-Seauencing)

Fifty thousand (50,000) Jurkats or CD8 T cells were harvested in PBS, centrifuged and transposase reaction was performed with standard Illumina reagents (e.g., TDE1/TD buffer, small kit; 20034197) according to the manufacturer's protocol. PCR cleanup was performed with MinElute column (Qiagen) followed by PCR amplification with qPCR guidance to avoid over-amplification (primers from Buenrostro et al, Nature Methods). Library purification was then performed using MinElute column and libraries were assessed by Agilent Bioanalyzer with high-sensitivity DNA kit.

### OPCR

One (1) μg of RNA was made into cDNA library using random hexamers. Hobit gene expression was confirmed using taqman probes and quantstudio qPCR machine.

### Cyclic Immunofluorescence Imaging

Cyclic immunofluorescence imaging (CyCIF) was used to investigate spatial context of infiltrating ZNF683 expressing cells in RT responders. Slides were examined from 3 baseline tumor biopsies (1 bone marrow, 2 lymph nodes) from the discovery cohort described herein.

Image data was processed and analyzed using CyCIF. Cells were segmented, intensities quantified and visualized in t-SNEs like other high-dimensional data.

### Example 2: Identification of Complex Populations in RT Bone Marrow

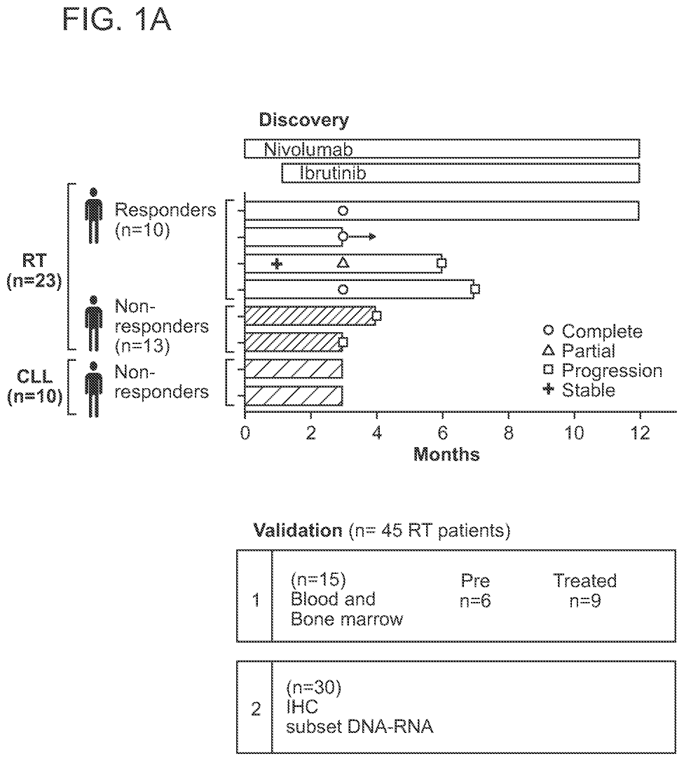

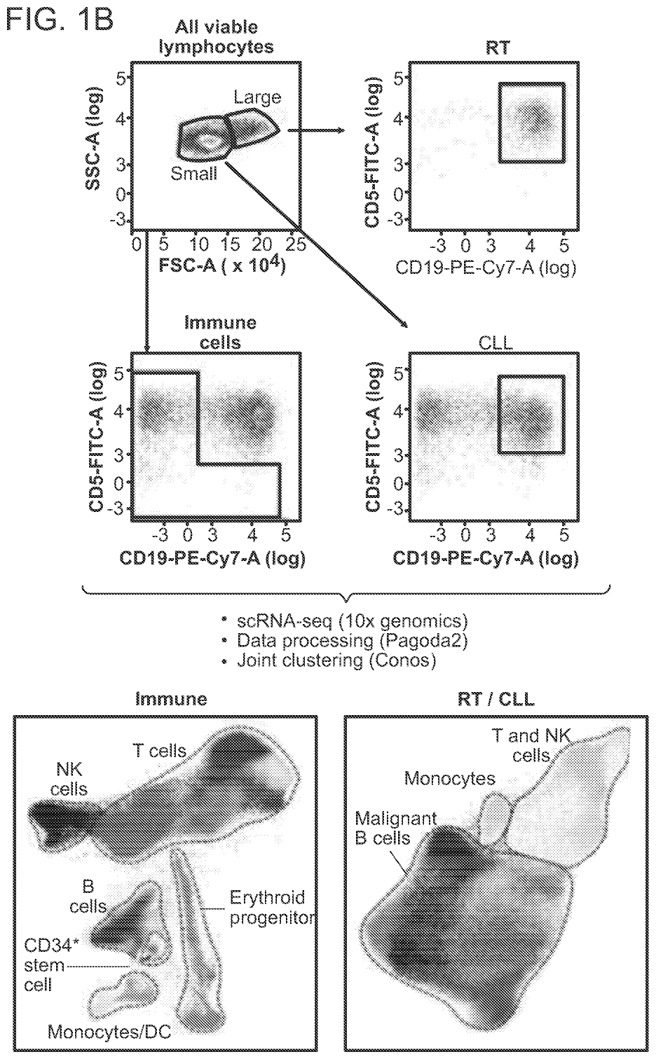

To systematically study cell populations associated with PD-1 CPB response, serial whole bone marrow (BM) samples collected from patients with RT or relapsed/refractory (R/R) CLL on an investigator-initiated phase II trial, treated with PD-1 blockade (nivolumab; 3 mg/kg every 2 weeks) plus ibrutinib (420 mg daily, continuous, starting day 1 of cycle 2)(NCT 02420912) were examined. Of 23 subjects with RT enrolled, 10 (ORR 43%) demonstrated response. In contrast, none of the 10 R/R CLL patients enrolled demonstrated benefit over single-agent ibrutinib. Discovery efforts were focused on a cohort of 6 patients with RT (4 responders, 2 non-responders) and two patients with R/R CLL. All RT responders achieved best response by month 3 although evidence of response was seen at initial 1 month response assessment in all responders aside from RT R3, which had initial stable disease followed by a delayed partial response by month three. Of these, 1 RT patient had pathologically confirmed marrow involvement at time of study initiation while all participants had detectable marrow CLL involvement and nodal RT. RT responders had prior CLL-directed chemotherapy exposure (range 1-4 lines of CLL therapy) as compared to NR, one which had novel agent exposure and the other only had RT-directed therapies. To provide the opportunity for confirming the insights gleaned from the discovery cohort, specimens from an independent cohort of 11 RT study subjects who had response evaluation, as well as 25 non-trial RT patients were analyzed (FIG. 1A).

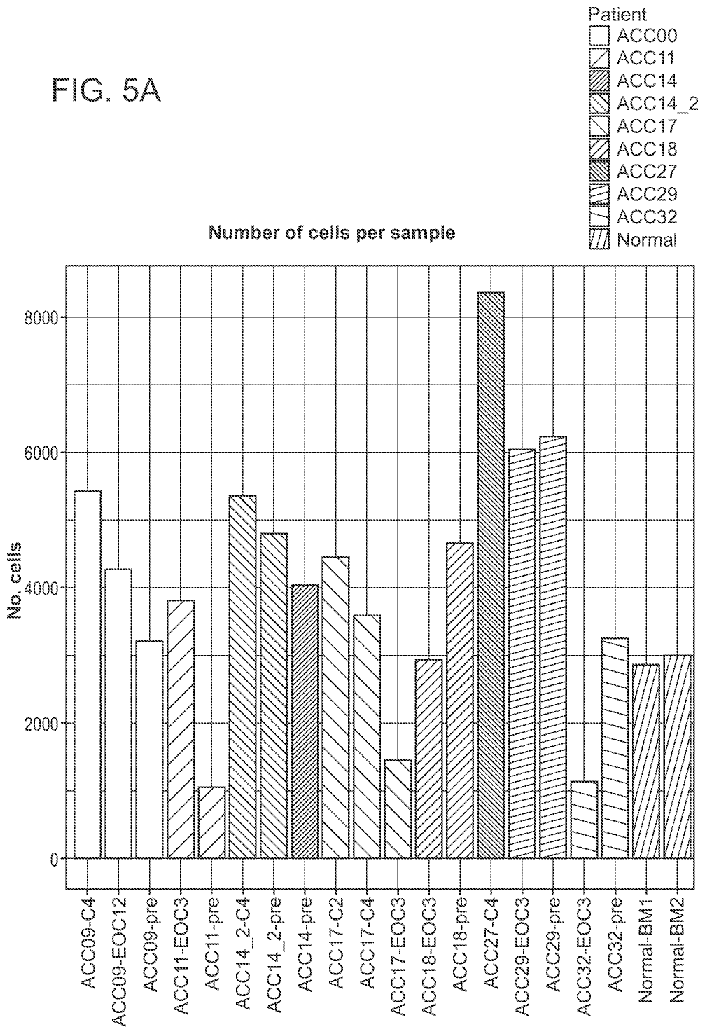

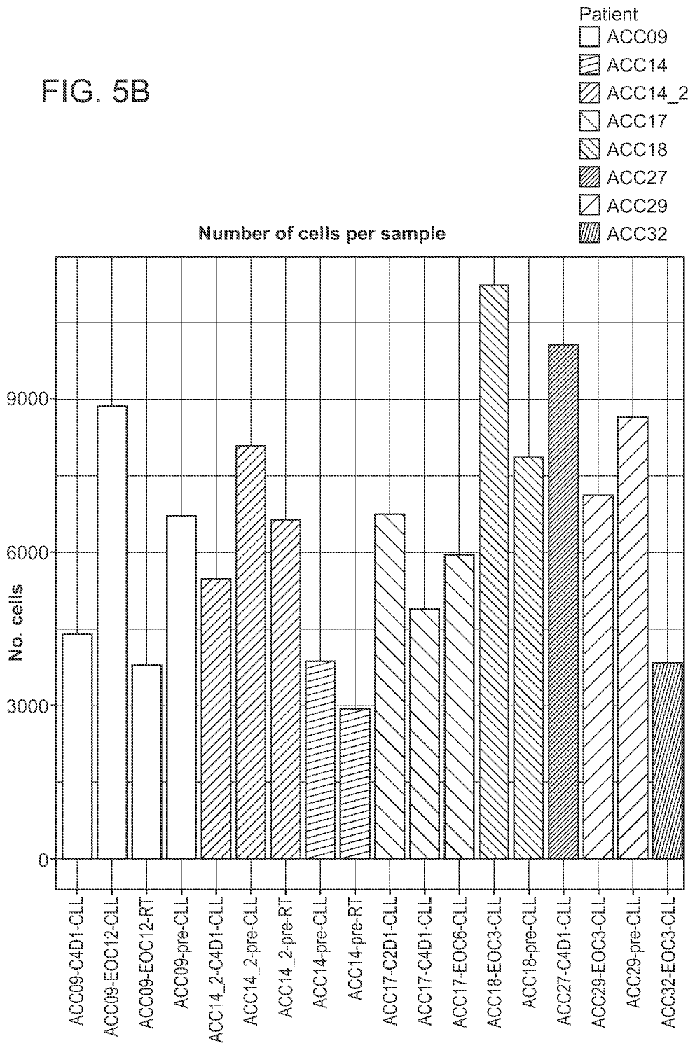

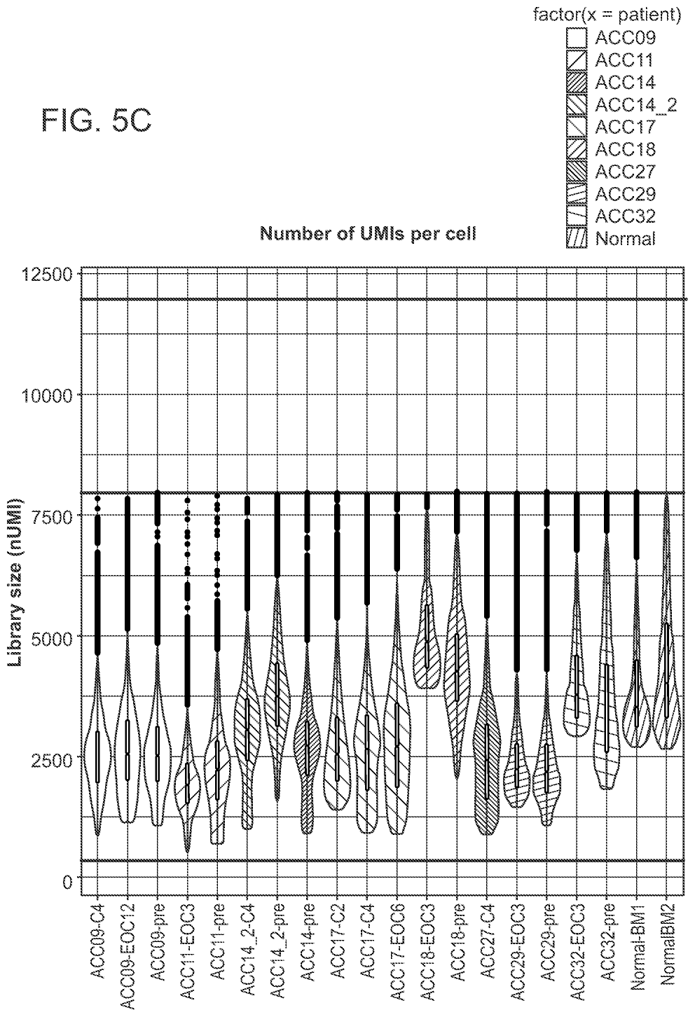

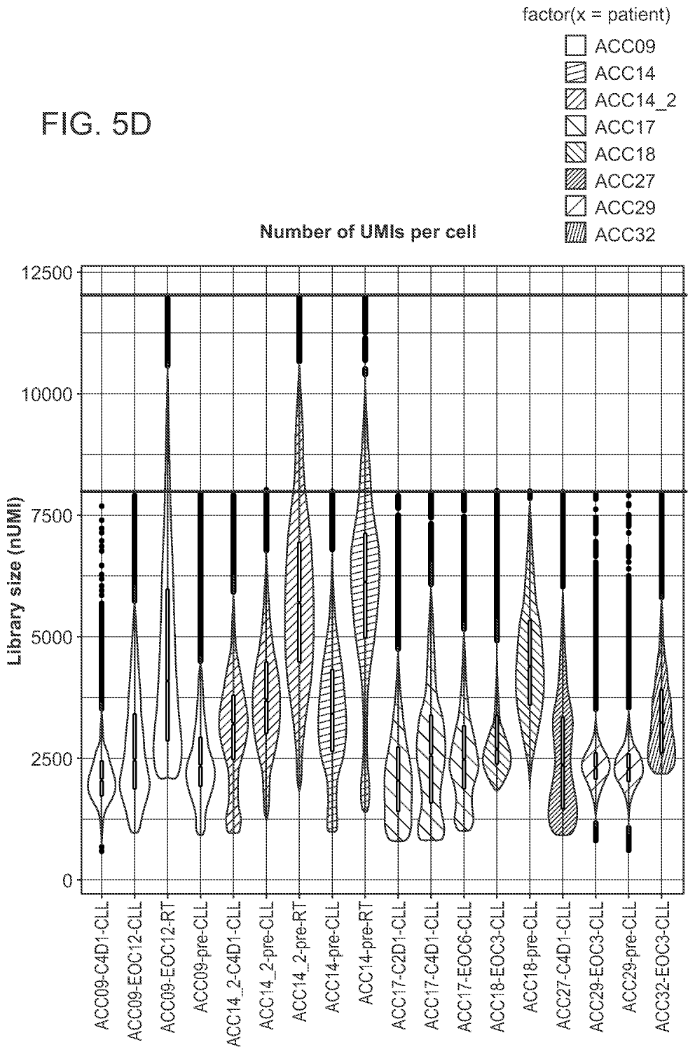

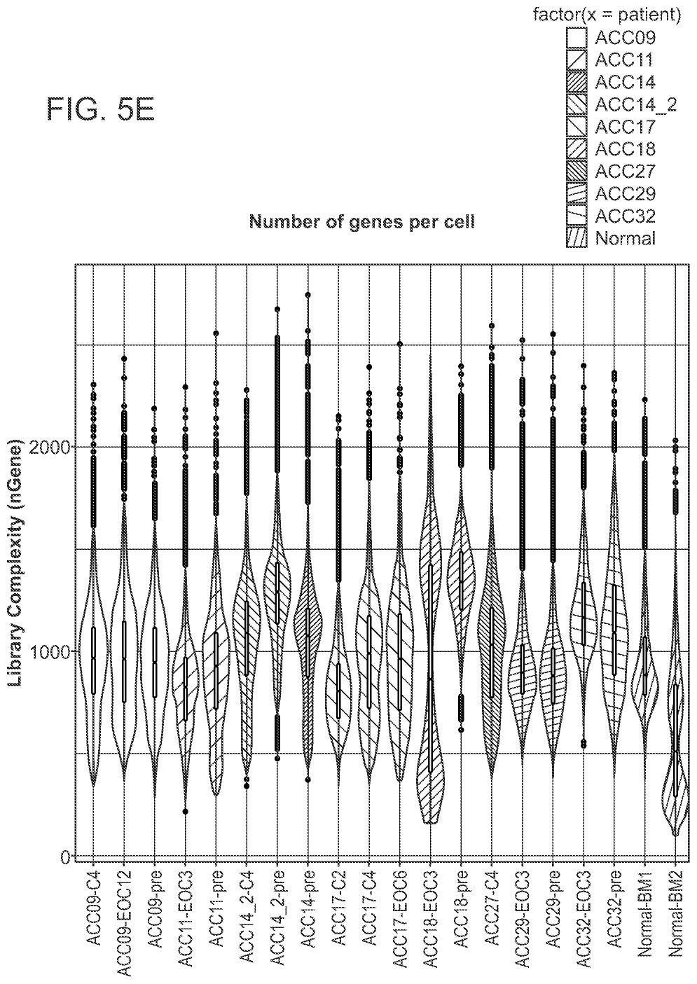

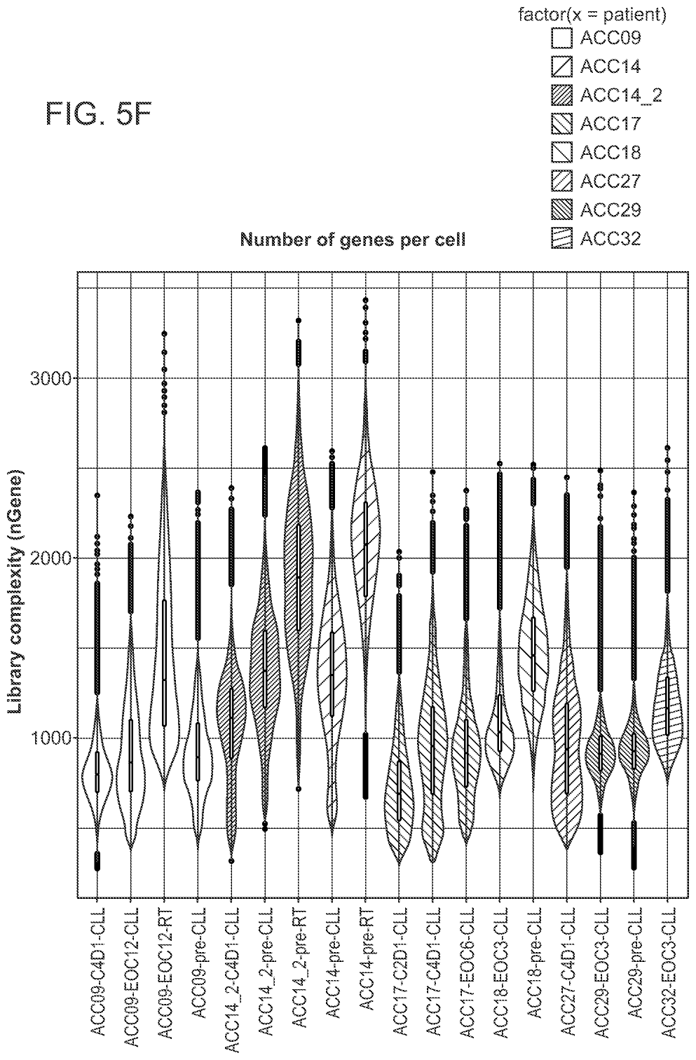

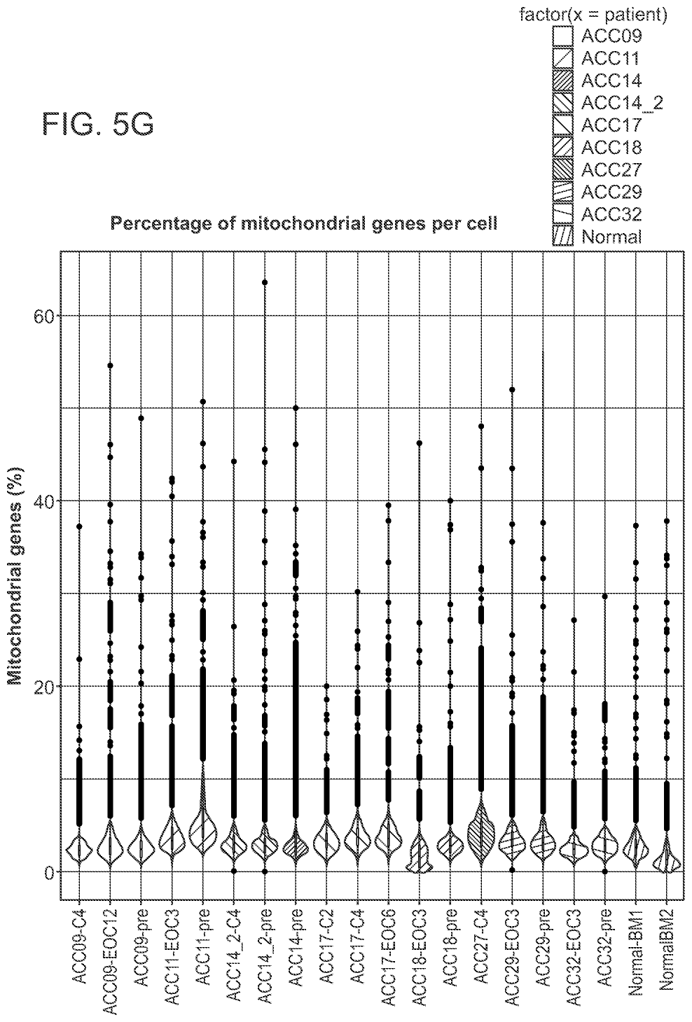

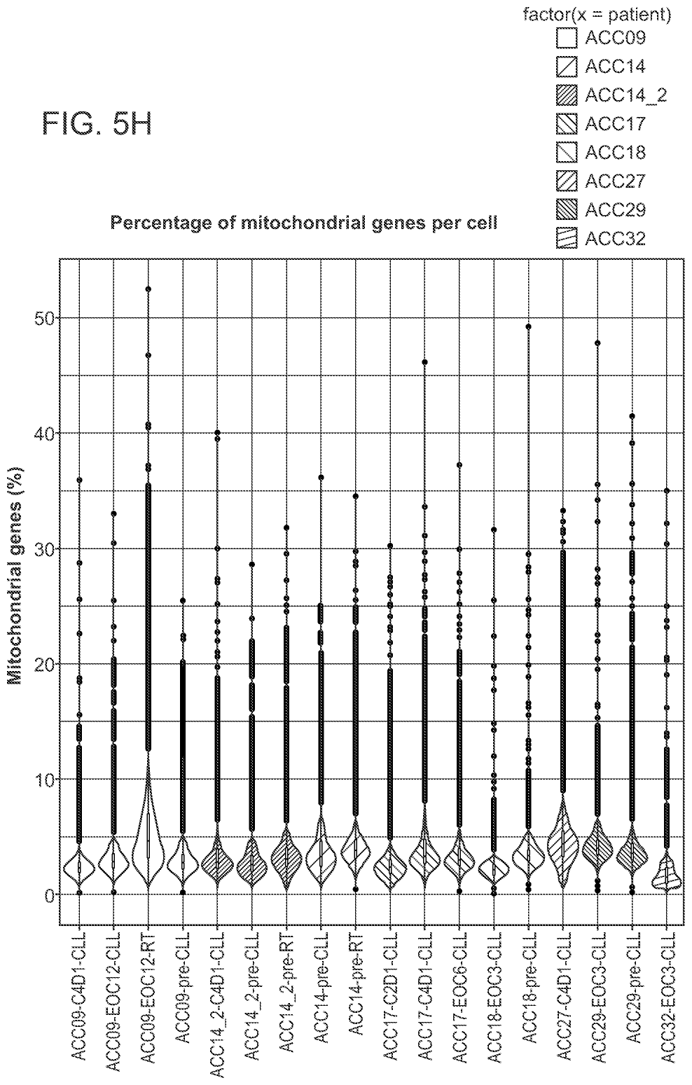

Samples were separated by fluorescence activated cell sorting (FACS)into viable tumor (CD5+CD19+) and non-tumor (CD5−CD19−) fractions, with further separation based on size (FSC/SSC) to identify RT cells (large) and CLL cells (small) when possible (FIG. 1B). To examine these heterogeneous populations at high resolution, single-cell RNA-sequencing was performed on these sorted marrow populations (Example 1). After initial filtering, joint clustering with Conos was performed on 78,488 non-tumor and 117,703 malignant cells from a total of 17 serial marrow samples, including marrow mononuclear cells from 2 age-matched healthy marrow donors in the non-tumor clustering (FIG. 1B). The captured average genes/cell are illustrated in FIG. 5A-FIG. 5H.

### Example 3: Immune Cell Populations Differ Between RT Responders and Non-Responders

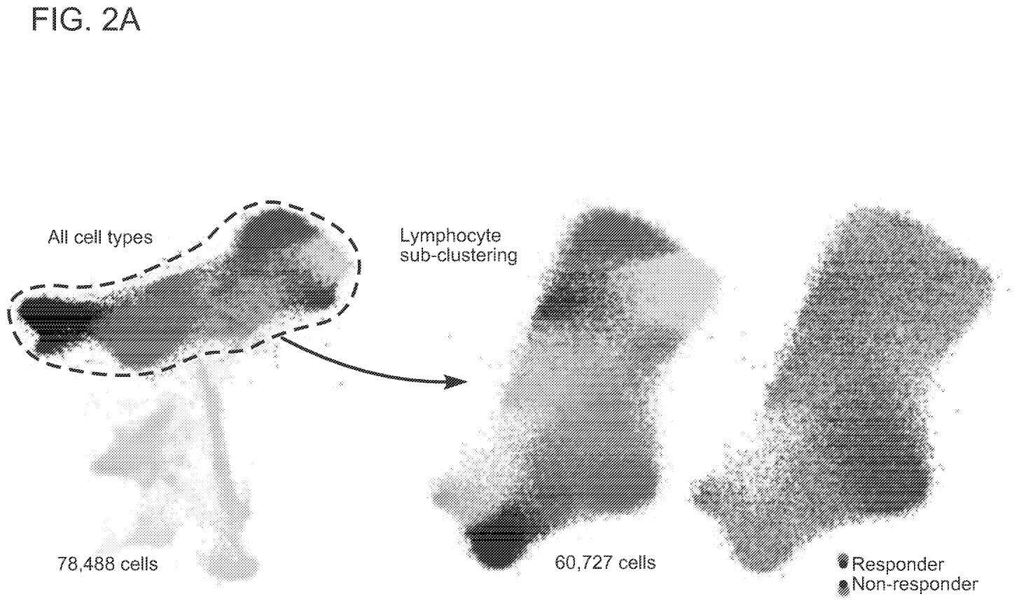

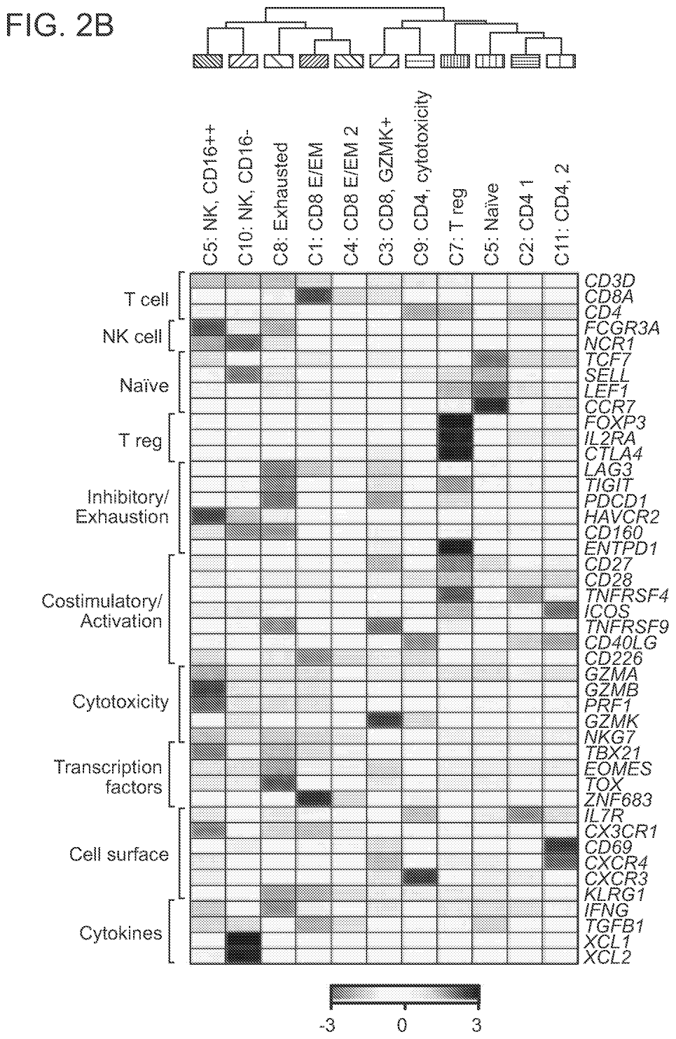

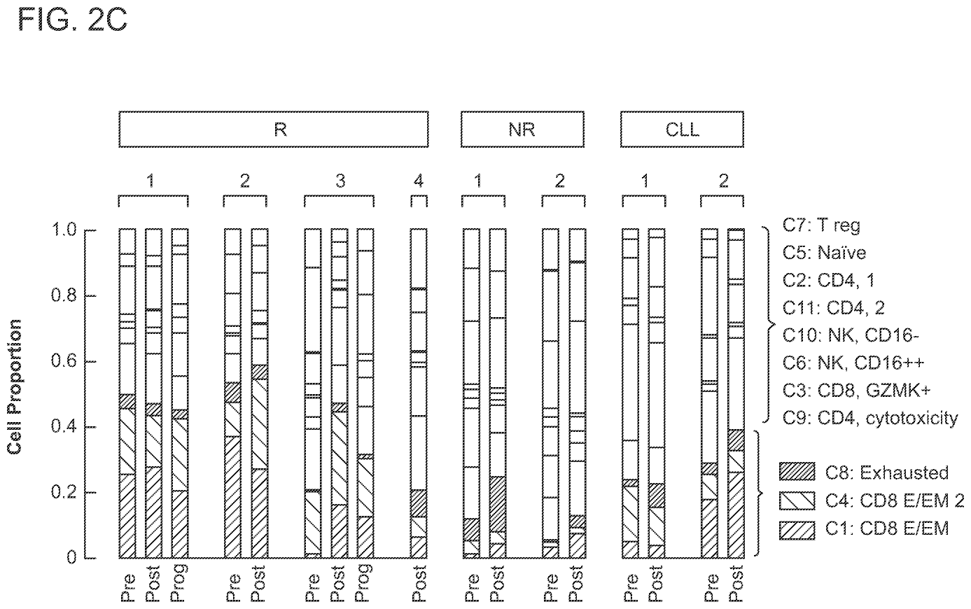

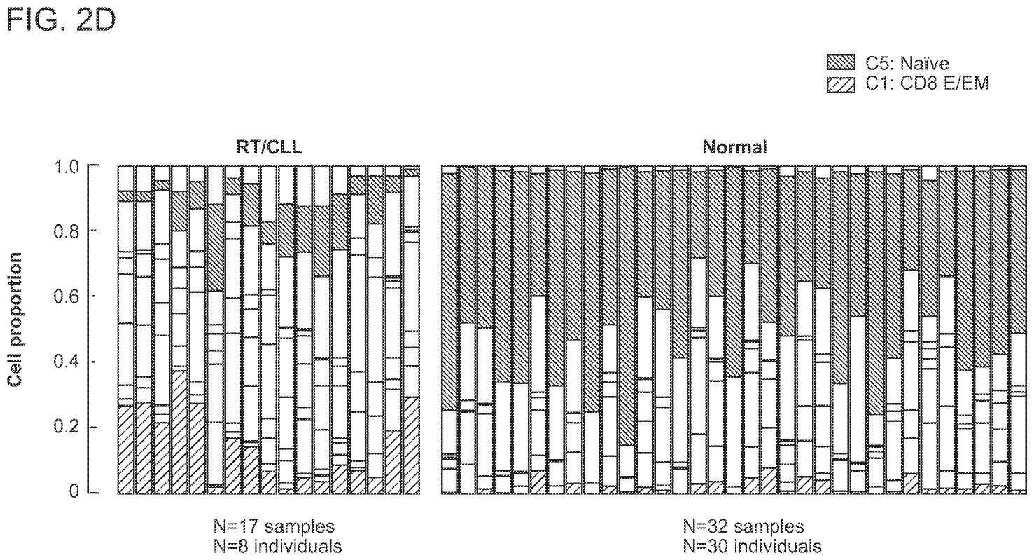

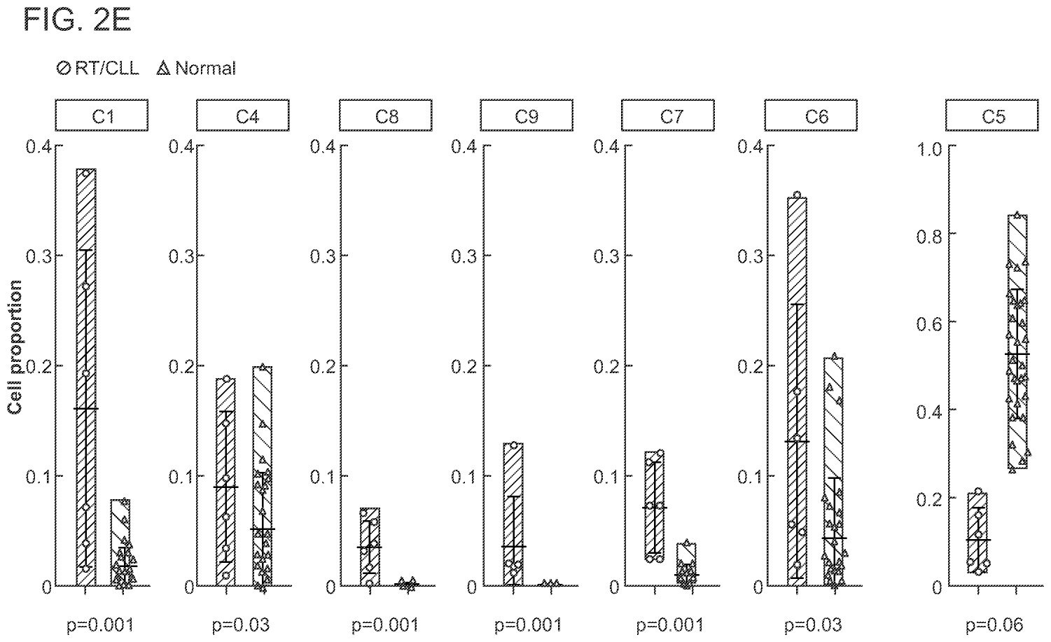

Given that the majority (77%) of marrow infiltrating non-tumor cells were lymphocytes (60,727 cells), the study was focused on the characterization of the diverse T and NK cell populations through sub-clustering was using Conos was (FIG. 2A). On average, 1001 genes/cell were captured for T and NK cells. A total of 11 transcriptionally distinct clusters were identified, consisting of 2 NK cell clusters (clusters 6 and 11) and 9 T cell clusters.

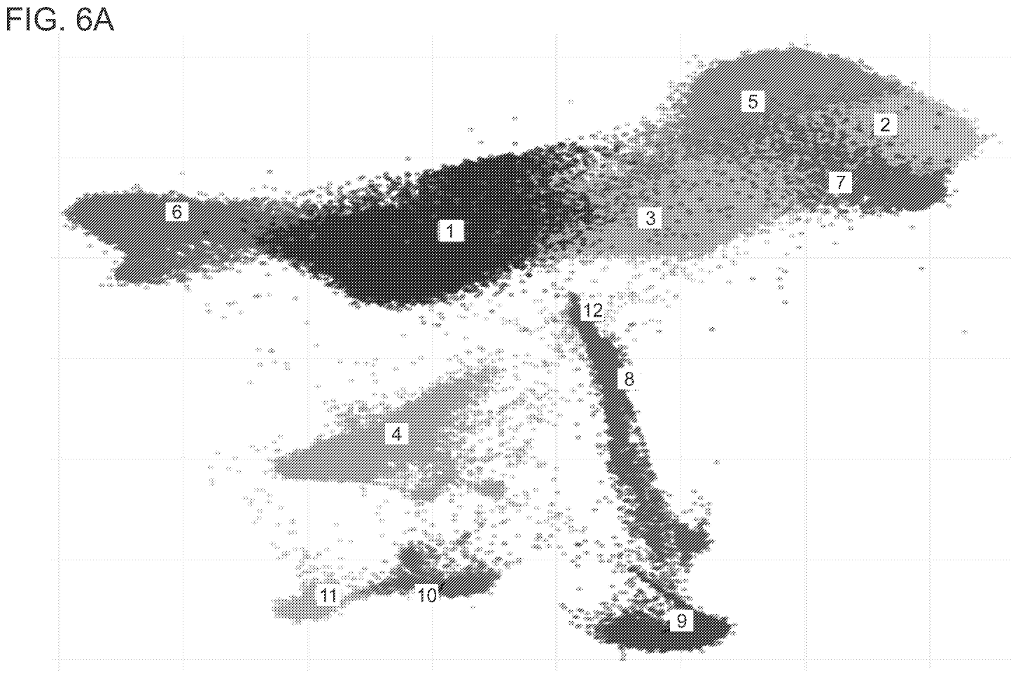

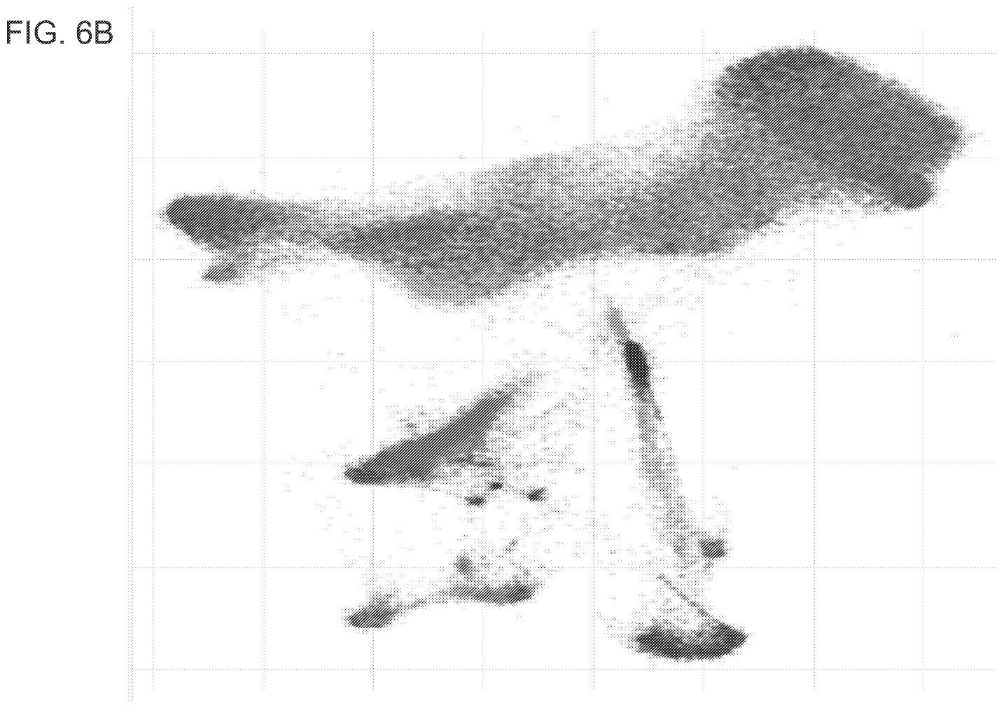

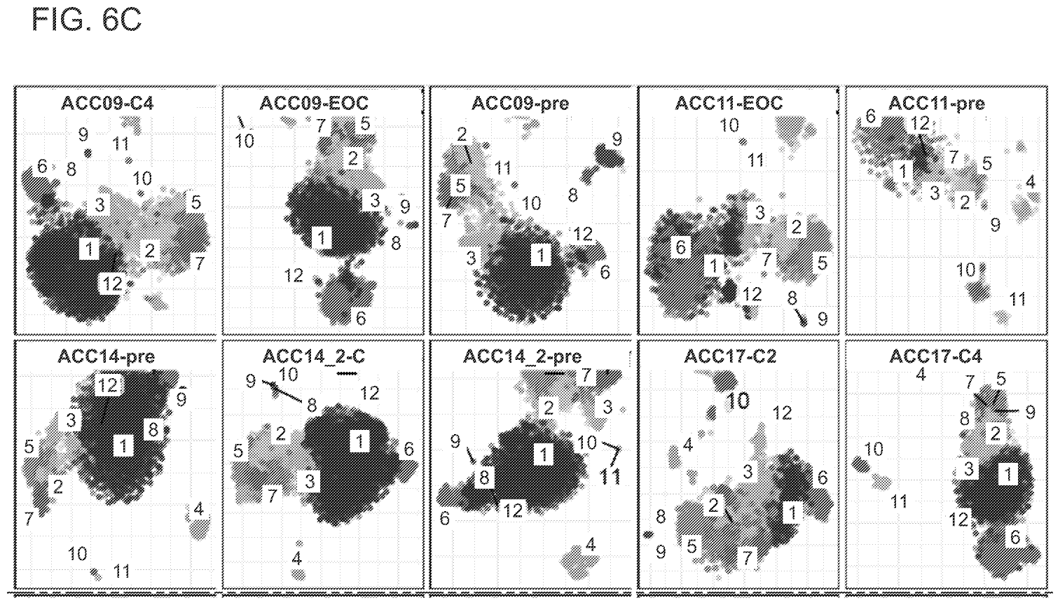

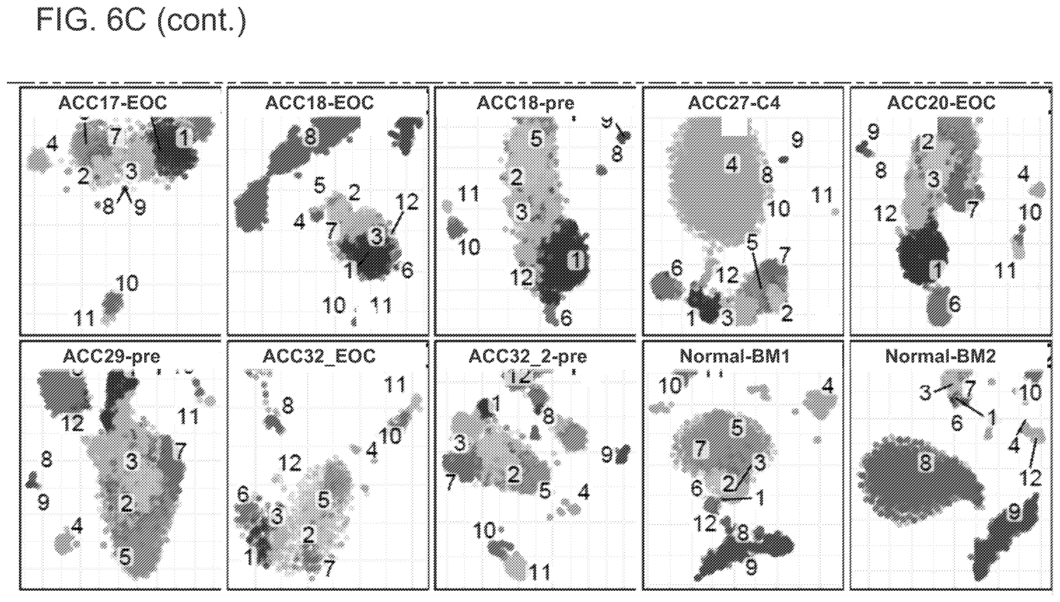

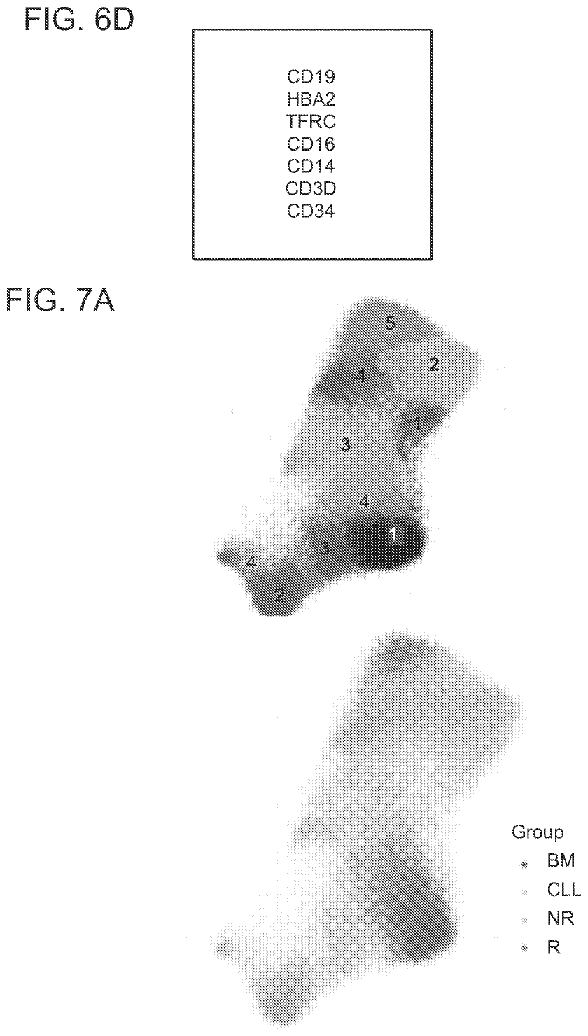

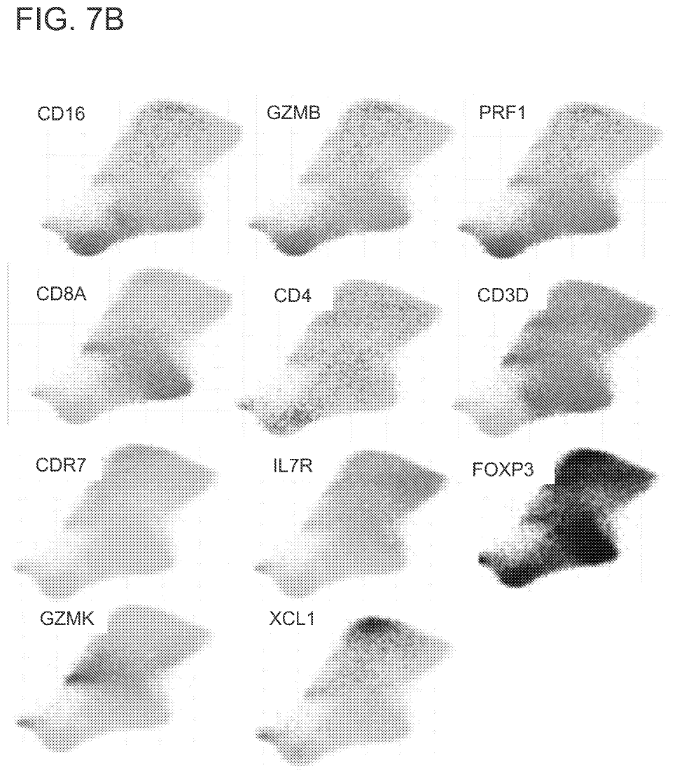

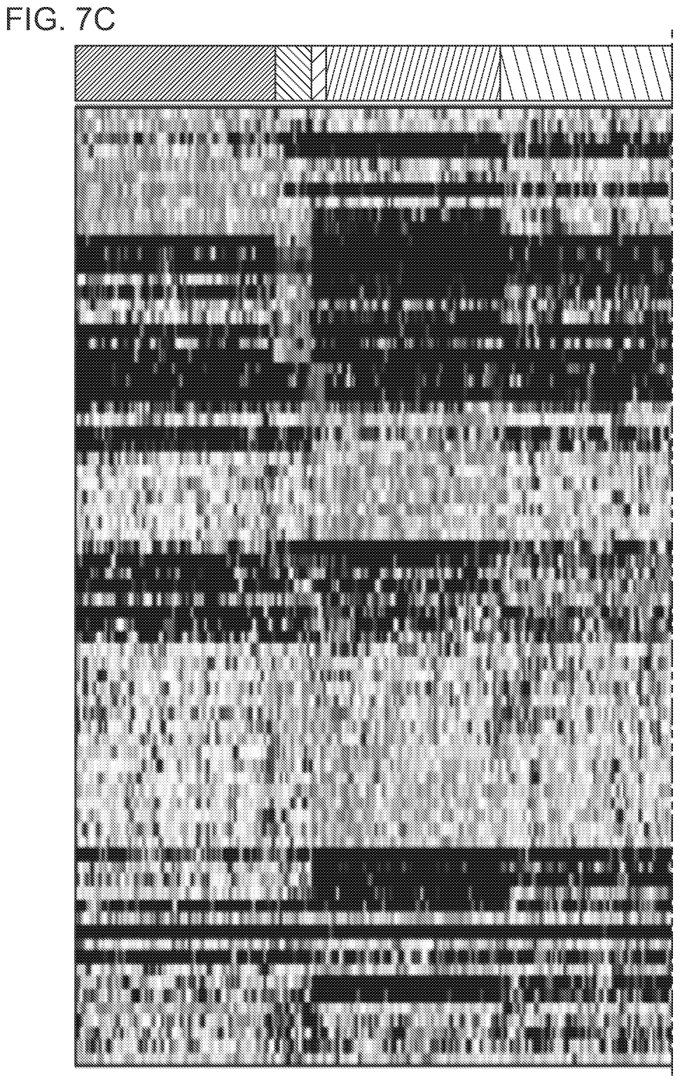

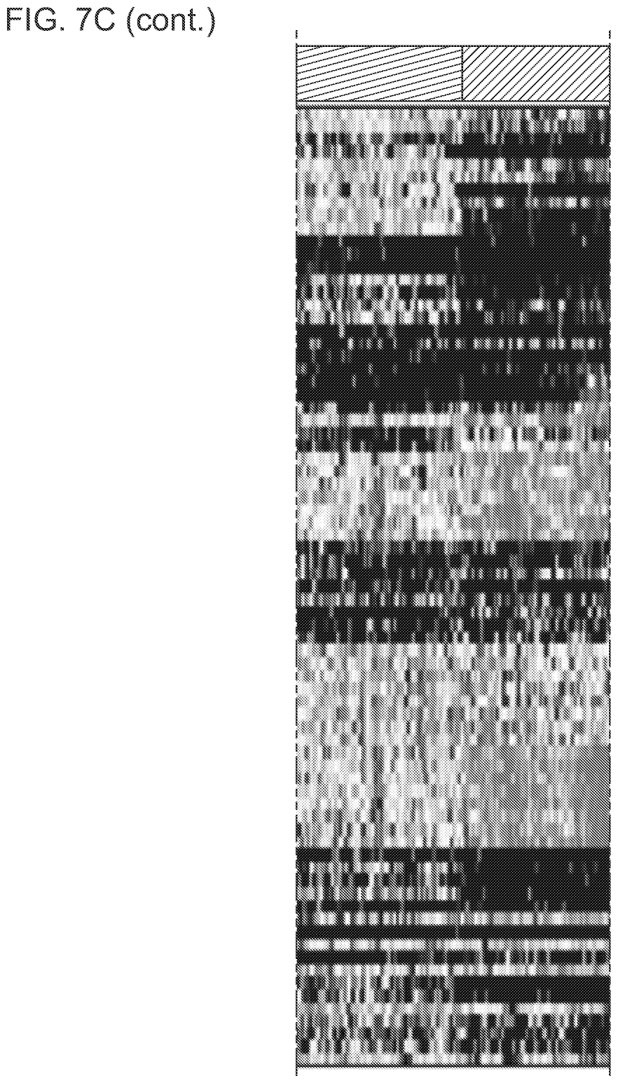

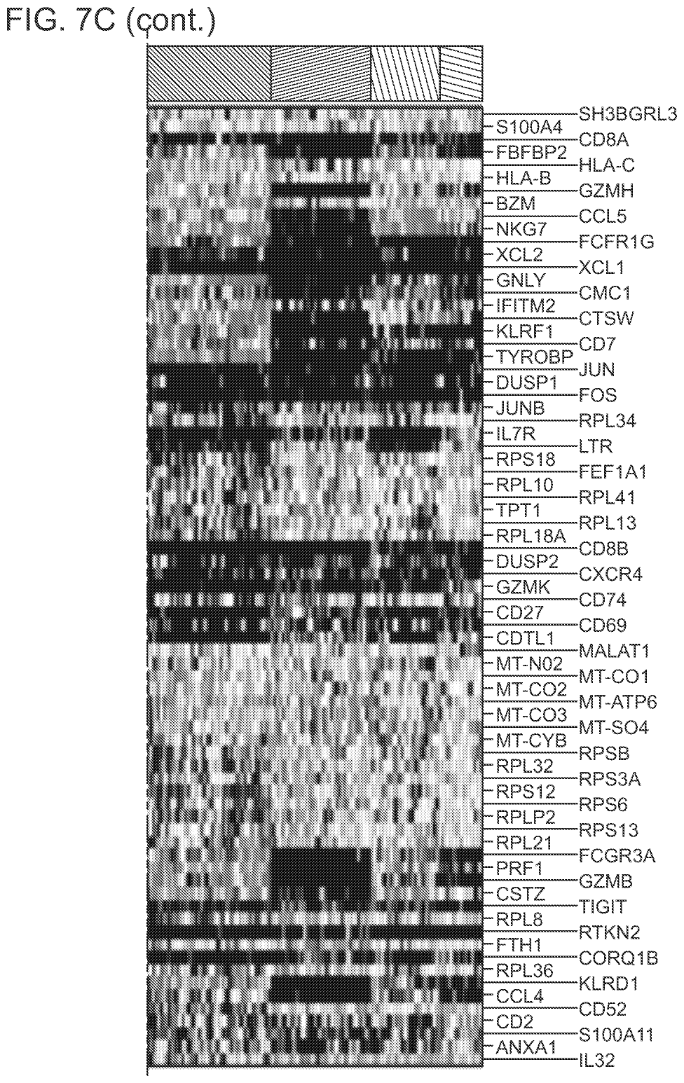

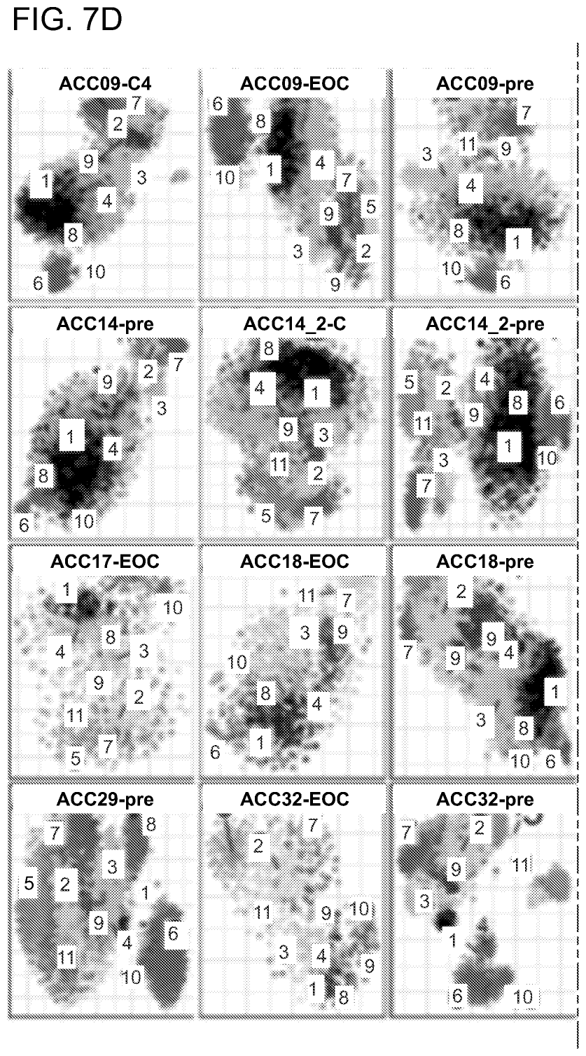

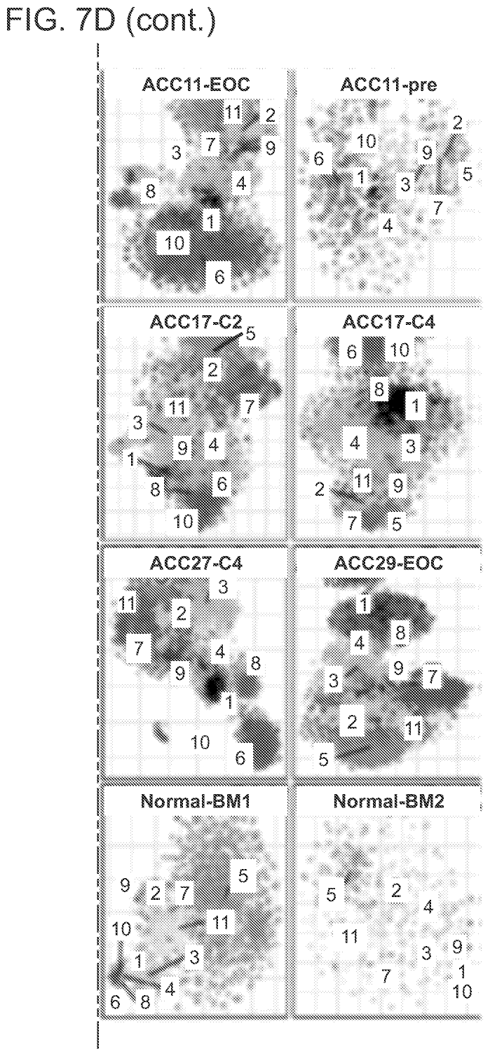

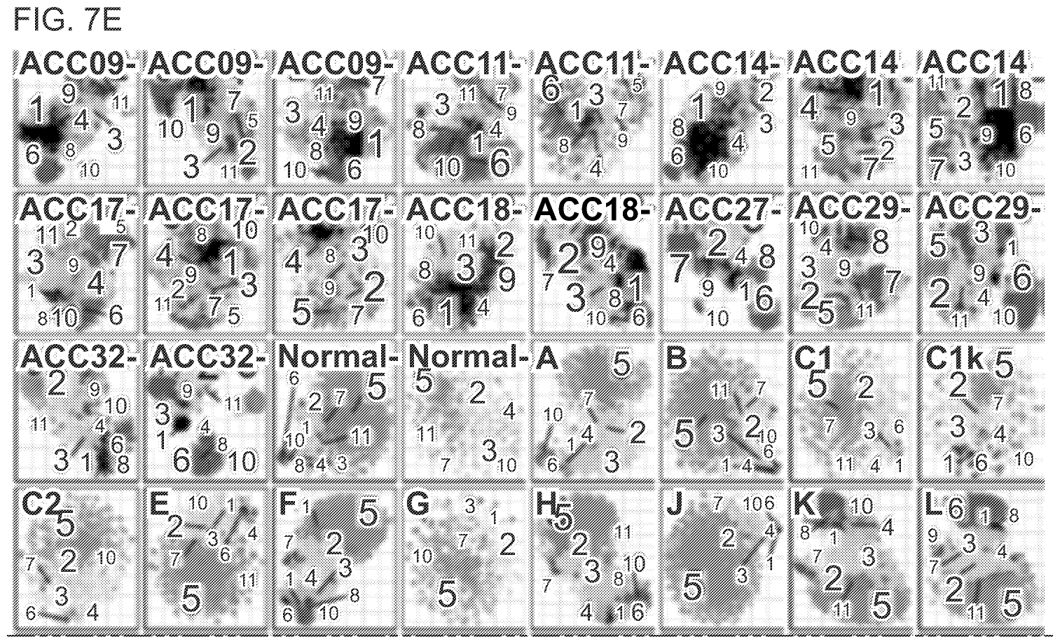

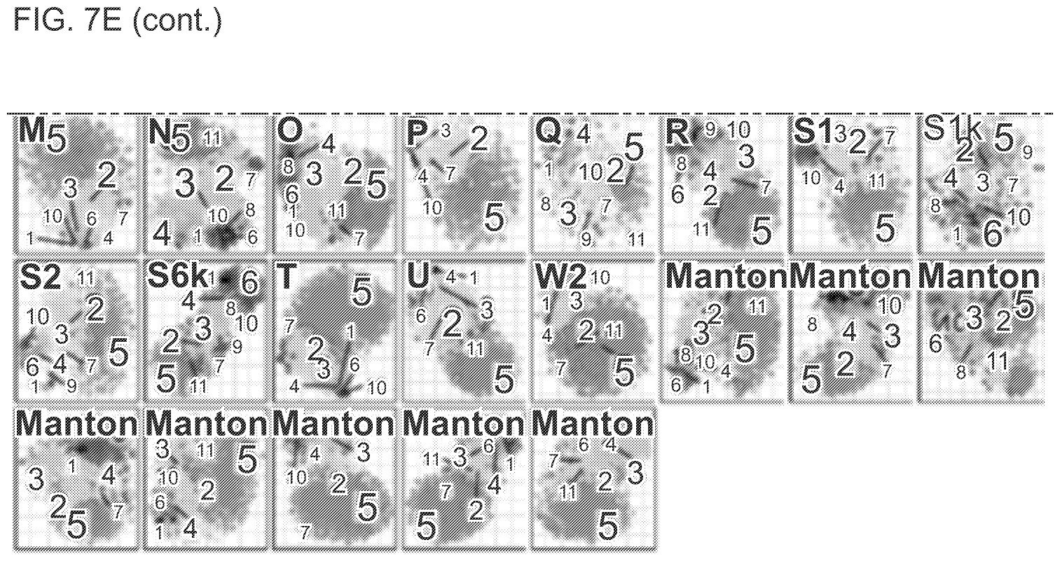

Transcriptionally defined clusters were identified based on a combination of cluster marker genes and lineage markers (FIG. 6A-FIG. 6D; defining genes highlighted in FIG. 2B). The differentially identified populations included Cluster 1, a large CD8 effector/effector memory (E/EM) cell population marked by the expression of cytolytic machinery genes and the transcription factor ZNF683. Cluster 1 demonstrated co-expression ofKLRGJ, CX3CR1 and LAG3 and low levels of other exhaustion/inhibitory markers (CD160. CD244). Cluster 3 consisted of CD8 T cells marked by GZMK gene expression and expressed genes suggestive of marrow resident memory (CD69. CXCR4) as well as the co-stimulatory marker CD27. A subset of Cluster 3 contained TCF7⁺ CD8 T cells. Cluster 4 contained a highly similar population but with slightly higher mitochondrial content and intermediate features between Cluster 1 and Cluster 3, such as lower GZMB. GZMA and PRFJ and higher IL 7R and GZMK compared to Cluster 1; Cluster 4 thus appears to be a transitional state between cells with high effector potential and Cluster 3. Other cytotoxic T cell populations included Cluster 8, which contained CD8 T cells with multiple exhaustion markers (TIGIT PDCDJ, LAG3. HAVCR2) and high TOX expression and Cluster 9, which included CD4 T cells with cytotoxic gene expression. NK cells segregated into two populations as previously described (ref), CD16^{high} NK cells with intense cytotoxic gene expression and CD16^{low} population marked by high expression of the chemokines XCL1/2. A naïve-like T cell population (expressing TCF7, LEF1. SELL and CCR7) consisting mainly of CD4 T cells and a small population of CD8 T cells formed Cluster 5. Cluster 7 showed features consistent with a T regulatory cluster (FOXP3. IL2RA. CTLA4. TIGIT). Clusters 2 and 11 were CD4 T cells. In subsequent CITE-seq experiments on marrow samples from RT-R2 and RT-NR1, in which 54 antibodies detecting cell surface markers, including three isotype controls, were co-analyzed with transcriptomes, thereby linking gene and protein expression data (10×5′), the identity of clusters was confirmed (FIG. 7A-FIG. 7E—CITE-seq).

### Example 4: Cytotoxic Marrow Populations are Enriched in RT and R/R CLL

These marrow lymphocyte clusters were compared to populations in normal bone marrow. Through examination of joint clustering results, the two normal marrows included in the initial clustering showed distinct differences as compared to RT and CLL marrows (FIG. 2D-FIG. 2E), including dramatic reduction in Cluster 1 and other cytotoxic populations. To assess the prevalence of the identified populations in normal bone marrow, 28 additional marrow mononuclear cell samples from publicly available 10× data (Oetjen et al., 2018 JCI Insight, 6;3(23):e124928) were examined. Conos joint clustering was performed on marrow lymphocytes with label propagation of our 11 lymphocyte clusters. Striking differences were observed (FIG. 2D) with enriched cytotoxic populations (Cluster 1 p=0.001, Cluster 4 p=0.03, Cluster 8 p=0.001, Cluster 9 p=0.001 and Cluster 6 p=0.03) and T regulatory cells (Cluster 7 p=0.001) in RT and CLL marrow. In contrast, normal marrow was enriched in cells with a naïve-like signature (Cluster 5, p=0.06).

### Example 5: RT Responders Show Increased Cells in Cluster 1

RT samples showed a high proportion of cytotoxic populations, predominantly Cluster 1 and Cluster 4, with stability in marrow populations across time in RT responders (FIG. 2E), with the exception of RT R3. R3 was a patient with delayed response and notable expansion of Cluster 1 and Cluster 4 cytotoxic populations at the time of response. These observations suggested that this population is important in anti-tumor response and led to the examination of whether there may be a predictive baseline signature present in RT responders. In examining pre-treatment samples from RT patients, RT responders indeed demonstrated a larger Cluster 1 CD8 E/EM population (p=0.04, t test) and fewer T regulatory cells (Cluster 7) (p=0.006, t-test) than RT non-responders. In examining the change in cell proportion from paired pre and post PD-1 CPB cluster proportions, RT non-responders showed a loss in NK cells (p=0.04).

### Example 6: Distinct Gene Signatures of T Cells Associated with CPB Response

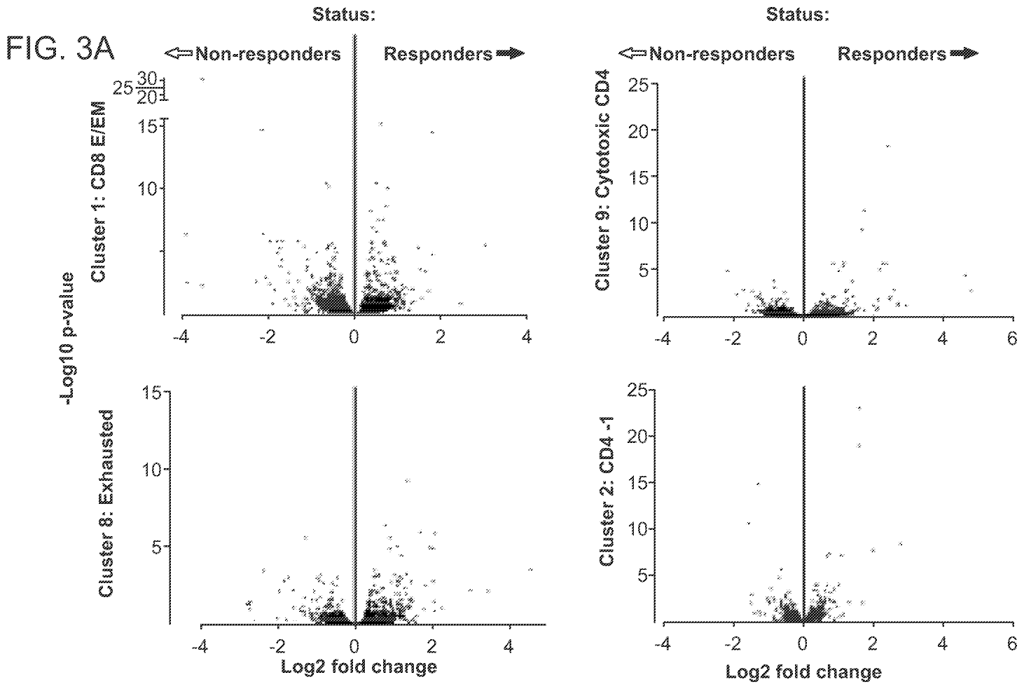

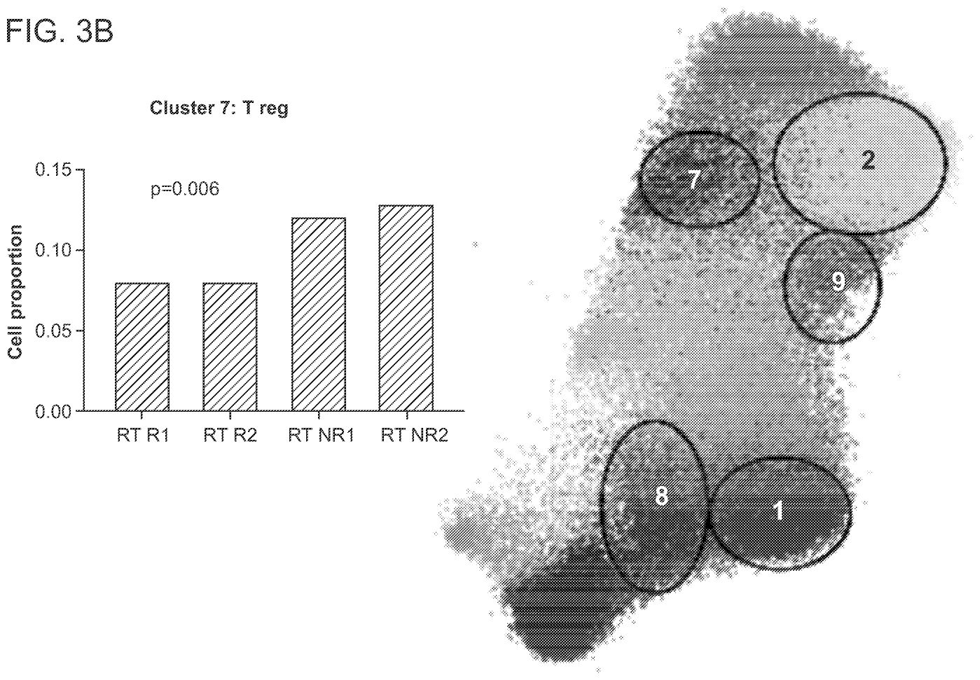

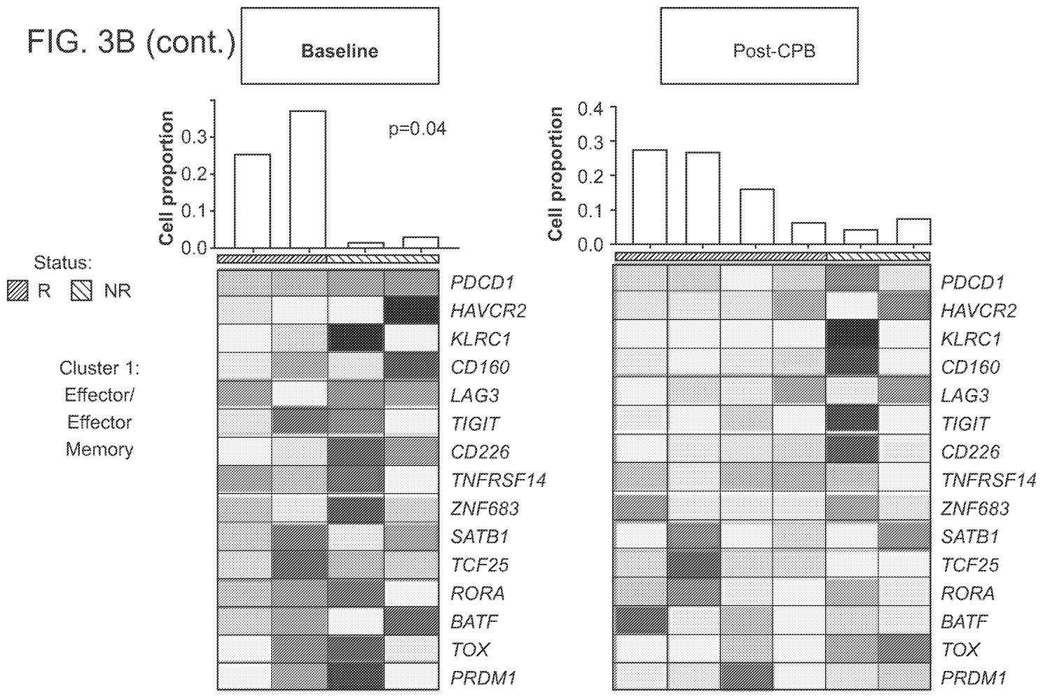

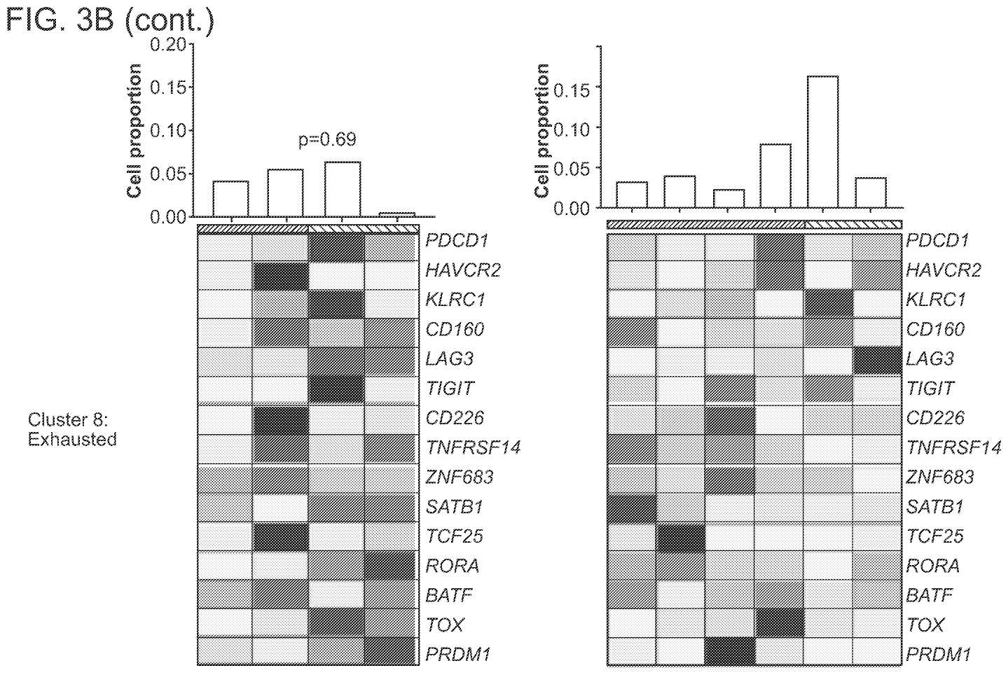

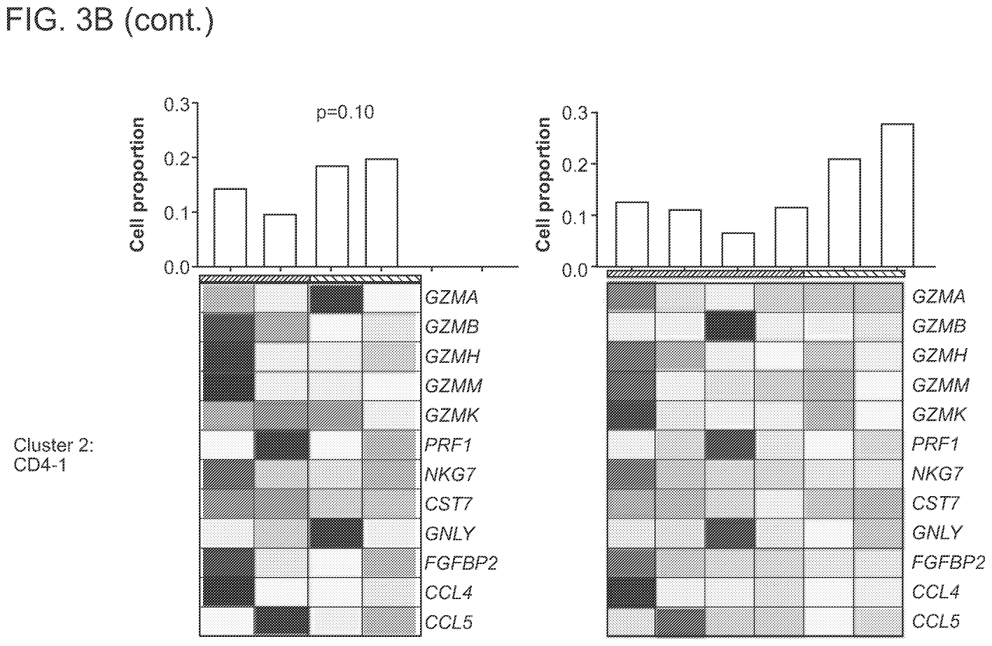

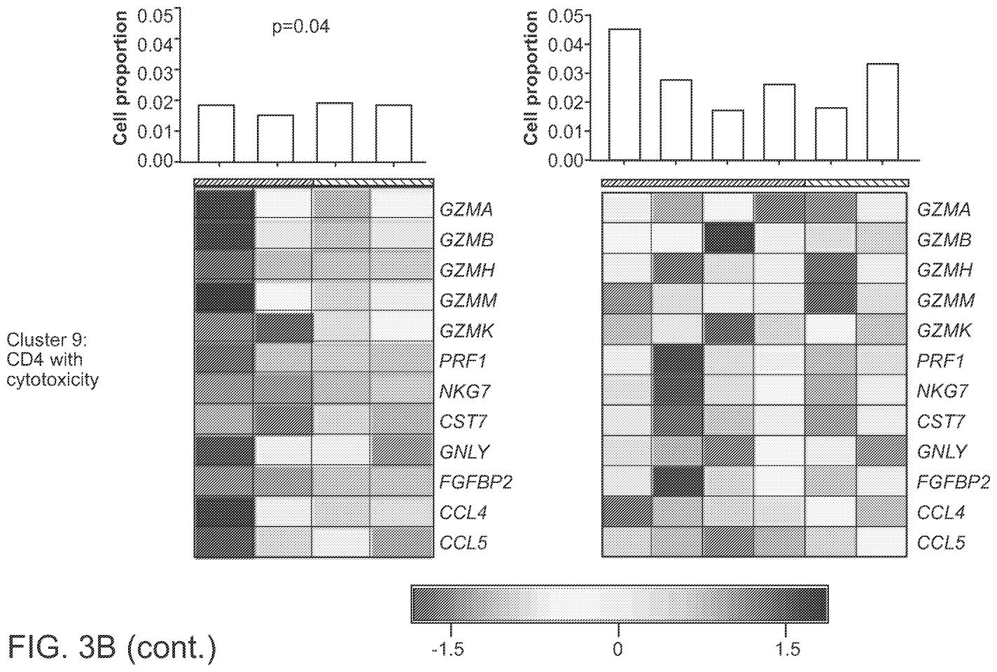

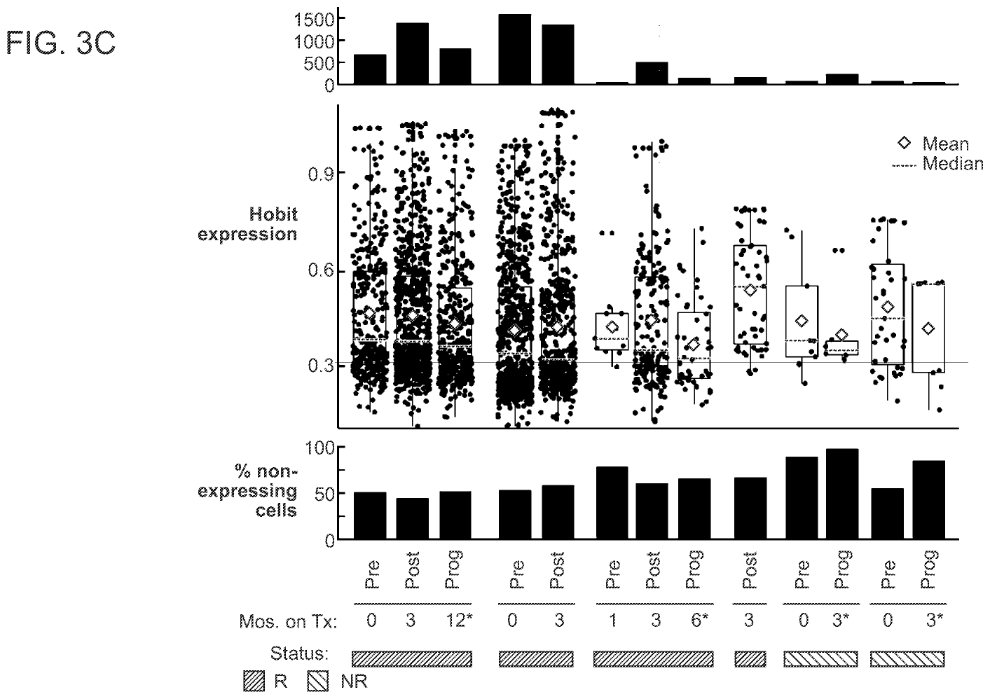

To assess for genes differentially regulated between RT responders and non-responders, differential gene expression analysis was performed on each cluster using DESeq2 with all pre and 3 month post-therapy samples, as this represented time of best response (FIG. 3A, FIG. 7A-FIG. 7E). In Cluster 1, some genes were differentially regulated between R and NR using a cutoff of log 2foldchange of >1 (or 0.5) and corrected p value of 0.05. This notably included several T cell transcription factors (RORA, TCF25 and BATF in R and TOX in NR) and immune signaling molecules (KLRC1, CD27, CD69, GZMK). In Cluster 8, —genes were differentially regulated with ZNF683, SATBI and CD226 higher in R. In the CD4 clusters 2 and 9, —and—genes were differentially regulated with higher expression of cytotoxic machinery (NKG7, CST7. PRFJ, GZMH) in RT responders. TCF7 did not vary significantly with response status in CD8 T cell clusters.

To determine whether these differences were present at baseline or time of response, the expression of T cell transcription factors, exhaustion markers and co-stimulatory genes were examined in baseline and 3 month response evaluation samples (FIG. 3B) from Cluster 1 and 8. This demonstrated that in addition to a quantitative increase in Cluster 1 cells, T cells from RT responders maintained high ZNF683 expression post-PD1 blockade. In contrast, T cells from RT non-responders showed increased TOX and PDCD1 in both pre and post therapy samples. In examining these same markers in the exhausted Cluster 8, ZNF683 expression was only seen in responders, as was CD226 expression. In the CD4 clusters 2 and 9, expression of granzymes, perforin, and chemokines was seen in responders relative to non-responders.

Furthermore, the genes that changed the most between pre and post therapy were examined independently in the 2 responders and 2 non-responders for which paired timepoint samples were available for analysis. RT non-responders upregulated cytotoxic genes in Cluster 1 (GZMB, PRF1, NKG7) but showed loss of ZNF683 (not significant). RT responders showed upregulation of inflammatory signatures.

### Example 7: ZNF683 is Increased in Responders and Maintained During Successful CPB Response

Given that ZNF683 both marked T cells of Cluster 1, which was enriched in responders, and was seen as differentially regulated when comparing the transcriptional profile of RT responders and non-responders, the expression of ZNF683 was examined in all our samples. Indeed, responders were clearly enriched in CD8 T cells in Cluster 1 with high expression of ZNF683 (by normalized count expression of ZNF683 and the percentage of cells expressing ZNF683; FIG. 3C). Furthermore, a dramatic expansion in ZNF683 expressing T cells appeared to be associated with response in RT-R3, the patient with delayed partial response. Providing additional evidence that the ZNF683 CD8 E/EM population is involved in response, this population appeared to decrease at the time of progression in both the 2 non-responders and in the 2 patients who progressed following initial response.

### Example 8: T Cell Clonotypes are Enriched in E/EM Cells in RT Responders

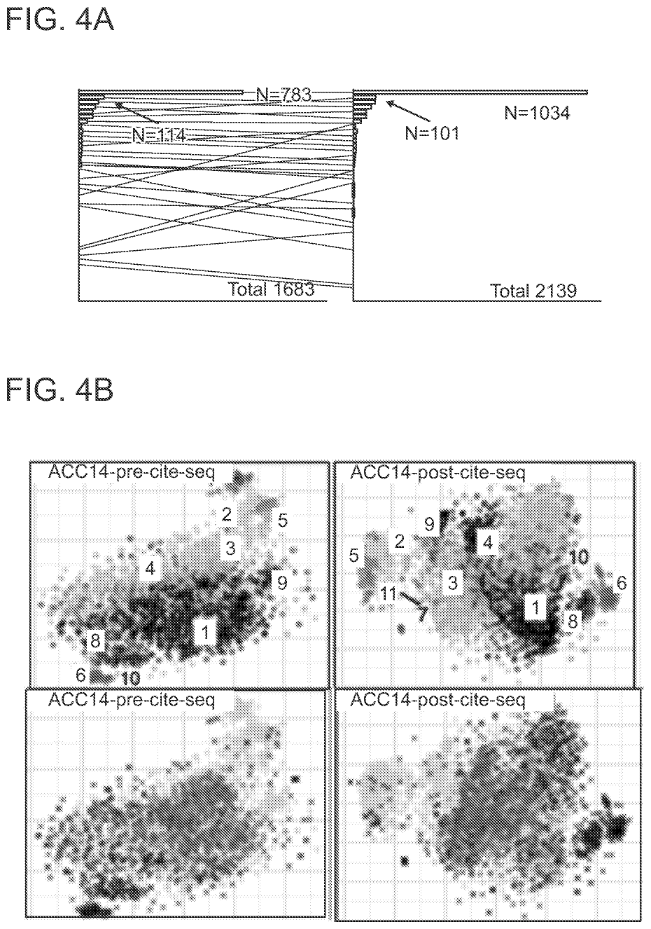

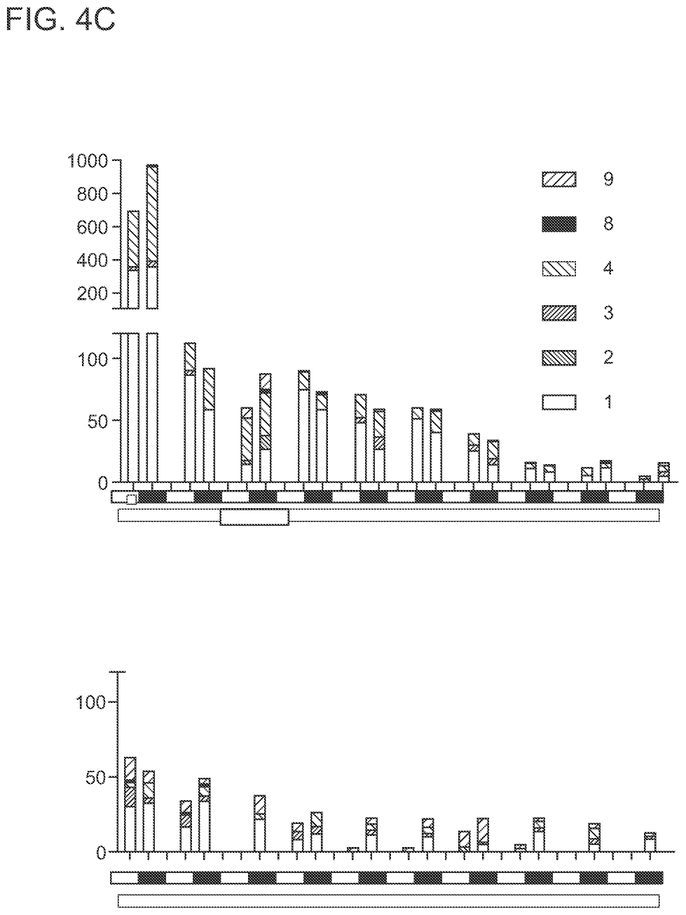

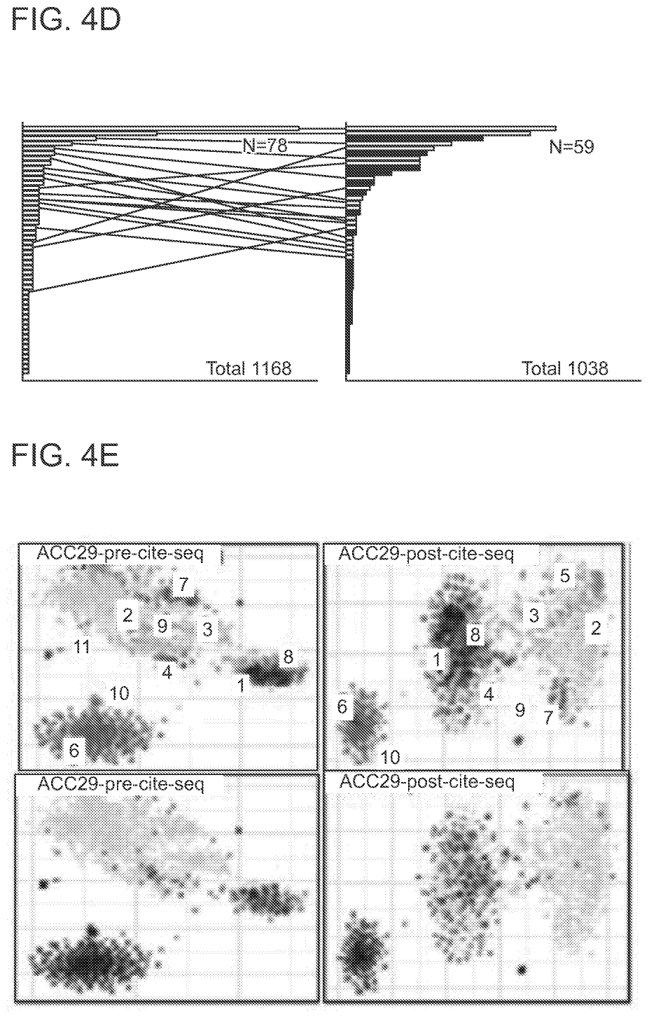

To link our identified T cell states with the T cell receptor (TCR) clonal repertoire, single-cell TCR sequencing along with gene and protein expression analysis (10×) were performed on an RT responder (R2) and RT non-responder (NR1) from which additional samples were available. The RT responder had dramatic clonal enrichment prior to PD-1 therapy and the top clones were still present post-therapy, suggesting tumor reactivity. Impressively, a dominant clonotype that was 43% of the productive clonal repertoire at baseline increased to 48% post-therapy. In contrast, the RT non-responder had increased T cell diversity, fewer enriched clonotypes and exhibited more clonotype expansion with PD-1 therapy (FIG. 4A). The T cell states of the top 10 most frequent clonotypes post-PD-1 blockade in both patients were then examined across time. T cell clonotypes spanned T cell clusters, demonstrating that T cell clones can exist in multiple different phenotypes in the bone marrow compartment. Interestingly, 9 of the 10 RT responder enriched clonotypes were CD8 T cell clonotypes that occupied Clusters 1, 4 and 3 (FIG. 4A). Thus, the CD8 clonotypes demonstrated co-existing ZNF683 expressing Cluster 1 phenotype with low level exhaustion/inhibition, a transitional state and memory, including some TCF7⁺ memory cells. The other enriched clonotype, clonotype 3, was a CD4 T cell that occupied Clusters 1, 4, 2 and 9 and thus consisted of cells with ZNF683 expression and high cytotoxicity and transcriptional profile shared with CD8 T cells and a unique CD4 cytotoxic population in addition to transitional and memory states. In contrast, the enriched clonotypes in the RT non-responder showed a high proportion of CD8 cells in exhausted clusters (p<0.001, poisson proportion test) in addition to cells with activated or memory phenotypes.

Notably, significant CD39 expression in CD8 cells was not observed, as this was largely restricted to T regulatory cells and CD103 expression was low-level throughout immune populations, highlighting major differences between markers of tumor-reactivity in tumor-infiltrating lymphocytes in solid tumors as compared to primary immune organs in hematologic malignancies.

### Example 9: T Cell Clonotypes Demonstrate Tumor Reactivity

To directly test tumor reactivity of enriched clonotypes from the RT responder, a modification of a transgenic TCR reporter system was used to overexpress the cloned TCRs with murinized constant regions in T cells pooled from healthy donors. These TCRs were then tested for reactivity against viral peptides from (EBV/FLU/etc.) and against autologous peripheral blood CD19+ CLL cells from time of RT remission transformed using EBV. Then, these TCRs were co-cultured with autologous tumor.

### Example 10: Enriched Clonotypes are Detectable Circulating in the Peripheral Blood

Bulk RNA-sequencing of paired peripheral blood from patients were analyzed as described in Example 1. The results are illustrated in FIG. 4D.

### Example 11: Profiling of ZNF683 and TOX Identify RT Responding Patients

Given the proposed association of ZNF683 expression and response to PD-1 blockade, ZNF683 protein was examined by flow cytometry in blood and bone marrow specimens from additional RT patients treated with PD-1 checkpoint blockade along with markers of T cell populations (CD3, CD4, CD8, CD45RA) and markers of exhaustion (PD-1, TOX).

To further evaluate the prevalence of these populations in abroad cohort of RT patients, multiplex immunofluorescence analysis of lymph node and bone marrow tumor biopsies was performed from RT patients and stained with 3 panels highlighting major immune populations. The study showed that the populations identified in bone marrow also can be detected in circulation and in the tumor microenvironment of the lymph node.

T cell transcription factor ZNF683 was identified as marker of a CD8 T cell effector/effector memory population with low level inhibitory markers that is unique to diseased marrow states and is enriched in RT responding patients. This population may represent a long-lived effector population with potential for immediate activation upon stimulation with PD-1 blockade anti-tumor specificity. Furthermore, this population also shares clonotypes with a transitional state and stem-like memory state with higher TCF7, linking this state with immediate effector potential to stem-like memory. However, differences in total TCF7 expression and response were not observed. Thus, the ZNF683 population may represent a deployment ready T cell arsenal that can respond readily to PD-1 blockade, as supported by the rapid kinetics of response of RT patients on clinical trial. Thus, this may be in contrast to solid tumors. Although ZNF683 high populations have been tied to increased survival in lung cancer. In contrast, T cells from non-responders lack notable ZNF683 expression or lose the expression upon treatment with PD-1 blockade in favor of a terminally differentiated more exhausted Temra state. Thus, this work suggests that the baseline pre-treatment T cell phenotypes determine response potential, at least in RT.

### Example 12: ZNF683 Infiltrating Cell Populations and Cell Contacts

To visualize the spatial context of infiltrating ZNF683 expressing cells in the RT responders, 3 baseline tumor biopsies (1 bone marrow, 2 lymph nodes) from our discovery cohort in high resolution were examined using CyCIF, or cyclic immunofloresence.

### Example 13: ZNF683 Overexpression Results

To probe the impact of ZNF683 transcription factor on T cell gene expression and chromatin state, a construct of ZNF683 and eGFP or eGFP alone were overexpressed in pooled stimulated T cells from 3 healthy blood donors using lentivirus. After culturing for ˜10 days, GFP-positive cells were sorted and then analyzed by RNA-seq and ATAC-seq. T cells were also phenotyped by flow cytometry.

### Example 14: RT Microenvironment/Tumor Factors

Concurrently with analysis of immune population, bone marrow malignant B cells for RT and CLL were examined to assess tumor-intrinsic contributions to response. Two patients had available RT for analysis and 7 patients had available CLL for analysis. RT cells showed both higher reads/cell (5,514 UMI/cell, 1,783 genes/cell, means for ACC09-EOC12-RT, ACC14_2-pre-RT, ACC14-pre-RT) compared to CLL (2,933 UMI/cell, 1,056 genes/cell, means for ACC09-C4D1-CLL, ACC09-EOC12-CLL, ACC09-pre-CLL, ACC14_2-C4D1-CLL, ACC14_2-pre-CLL, ACC14-pre-CLL, ACC17-C2D1-CLL, ACC17-C4D1-CLL, ACC17-EOC6-CLL, ACC18-EOC3-CLL, ACC18-pre-CLL, ACC27-C4D1-CLL, ACC29-EOC3-CLL, ACC29-pre-CLL, ACC32-EOC3-CLL), similar to as reported in acute myeloid leukemia (AML) (Zheng et al.). Using unsupervised joint clustering, RT also clustered separately from CLL and showed evidence of de-differentiation although B cell lineage markers and HLA expression were maintained. RT cells also showed upregulation of MYC signaling. Interestingly, the malignant B cells expressed a wide variety of inhibitory and immune modulatory molecules. However, these cells were not PD-1 or PD-L1 expressing. Upon treatment with PD-1 and ibrutinib, malignant CLL cells downregulated inhibitory signaling and HLA presentation and the surviving population was CXCR4 high. Thus, through inhibition of BCR signaling, ibrutinib decreases inhibitory signals in the microenvironment.

### Example 15: T Cell Determinants of Response and Resistance to PD-1 Blockade in Richter's Transformation

A discovery cohort of6 patients with RT (4 responders, 2 non-responders) and 2 patients with relapsed/refractory CLL enrolled on a study in which patients were initiated with anti-PD1 therapy (nivolumab 3 mg/kg every 2 weeks), with subsequent concurrent ibrutinib (420 mg daily)(NCT 02420912) was used in this study. A total of 15 serial study marrow specimens collected at treatment initiation and 3 month response evaluation, as well as 2 healthy marrow donors were examined.

To systematically discover the T cell populations and states associated with CPB response in RT, single-cell RNA-sequencing (scRNA-seq, 10× Genomics) of non-lymphoma (CD5−CD19−) cells isolated by flow cytometry from marrow samples was performed. A total of 60,727 T and NK cells were captured with average detection of 1001 genes/cell. Using the joint clustering approach Conos, 11 transcriptionally distinct clusters of lymphocytes were identified. Baseline RT/CLL with normal marrow and observed differences across T cell populations, which were confirmed through the examination of publicly available marrow scRNA-seq data from 28 healthy donors, were contrasted. Compared to normal marrow, RT/CLL marrow was enriched for cytotoxic populations, including both CD8 effector/effector memory (E/EM) (p=0.001, t-test) and cytotoxic CD4 (p=0.001) T cells as well as for cells expressing multiple exhaustion markers, including PDCD1, LAG3 and TIGIT (p=0.001). In contrast, normal marrow contained increased T cells with a naïve-like phenotype (p=0.06).

In the pre-treatment samples from RT patients, RT responders had a larger CD8 E/EM population (p=0.04) and fewer T regulatory cells (p=0.006, t-test) than RT non-responders. Using DESeq2 to compare clusters from all samples, differences in gene expression between RT responders and non-responders were evaluated. CD8 E/EM T cells of RT non-responders showed increased expression of TOX, a recently uncovered master regulator of cell exhaustion (padj=0.00016), while this cell subtype in RT responders upregulated a contrasting program of activating transcription factors as well as the co-stimulatory gene CD226(padj=0.04). As for CD4 T cells, RT responders revealed an enriched cytotoxic gene program compared to RT non-responders (padj PRF1 5.9×10-10, GZMH 6.0×10-6, NKG7 6.4×10-19).

To investigate whether response to CPB therapy for RT was associated with changes in the T cell receptor (TCR) repertoire, and to obtain protein-level validation of transcriptional signatures, single-cell TCR sequencing with paired gene and protein expression (10× Genomics) was performed on pre- and post-therapy samples from a RT responder and a non-responder. The gene expression findings, including validation of cytotoxic CD4 T cells and the enrichment of CD226 protein in E/EM CD8 T cells in the RT responder, were confirmed. TCR clonal expansion was observed in the RT responder at baseline with persistence of enriched clonotypes following CPB, suggesting the presence of tumor-reactive T cell clones. In contrast, the RT non-responder displayed higher TCR diversity with enriched clonotypes showing increased exhaustion post-CPB (p<0.001, Poisson rate test). Ongoing validation studies include characterizing the phenotypes of corresponding peripheral blood T cells and confirming our findings in an independent, larger cohort of RT patients using multiplex immunofluorescence and flow cytometry.

In conclusion, marrow T cell populations enriched in RT patients and distinct T cell transcriptional programs that delineate RT responders from non-responders are identified and described herein. Candidate gene biomarkers that may identify patients likely to respond to CPB therapies and a CD8 E/EM T cell population that is likely to underlie response to PD-1 CPB are described herein.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of treating a subject with Richter's transformation, comprising:
obtaining a test sample from a subject having or at risk of having Richter's transformation (RT);
determining the expression level of at least one RT-associated gene in the test sample;
comparing the expression level of the RT-associated gene in the test sample with the expression level of the RT-associated gene in a reference sample; and
determining whether programmed cell death protein-1 (PD-1) inhibition will inhibit RT and provide a clinical benefit to the subject if the expression level of the RT-associated gene in the test sample is differentially expressed as compared to the level of the RT-associated gene in the reference sample; and
administering a PD-1 inhibitor to the subject in whom the expression level of the RT-associated gene in the test sample is differentially expressed as compared to the level of the RT-associated gene in the reference sample.

2. The method of claim 1, wherein the test sample is obtained from bone marrow, a tumor tissue, a tumor microenvironment, a plasma sample, or a blood sample.

3. The method of claim 1, wherein clinical benefit in the subject comprises complete or partial response as defined by response evaluation criteria in solid tumors (RECIST), stable disease as defined by RECIST, or long-term survival in spite of disease progression or response as defined by irRC criteria.

4. The method of claim 1, wherein the RT-associated gene comprises Zinc finger protein 683 gene (ZNF683); and
determining that inhibition of PD-1 in the subject with RT will result in clinical benefit in the subject if the expression level of the ZNF683 gene in the test sample is higher than the level of the ZNF683 gene in the reference sample.

5. The method of claim 1, wherein the RT-associated gene comprises a thymocyte selection-associated high mobility group box protein gene (TOX); and
determining that inhibition of PD-1 in the subject with RT will not result in clinical benefit in the subject if the expression level of the TOXgene in the test sample is higher than the level of the TOXgene in the reference sample.

6. The method of claim 1, further comprising treating the subject with a chemotherapeutic agent, radiation therapy, cryotherapy, hormone therapy, or immunotherapy.

7. The method of claim 6, wherein the chemotherapeutic agent comprises dacarbazine, temozolomide, nab-paclitaxel, paclitaxel, cisplatin, or carboplatin.

8. The method of claim 5, further comprising administering an inhibitor of the RT-associated gene with a higher level of expression compared to the level of the RT-associated gene in the reference sample, thereby treating the RT.

9. The method of claim 8, wherein the inhibitor of the RT-associated gene comprises a small molecule inhibitor, RNA interference (RNAi), microRNA (miRNA), an antibody, an antibody fragment, an antibody drug conjugate, or any combination thereof.

10. The method of claim 1, wherein the PD-1 inhibitor comprises a small molecule inhibitor, RNA interference (RNAi), microRNA (miRNA), an antibody, an antibody fragment, an antibody drug conjugate, an aptamer, a chimeric antigen receptor (CAR), a T cell receptor, or any combination thereof.

11. The method of claim 10, wherein the PD-1 inhibitor comprises an antibody or antibody fragment, and wherein the antibody or antibody fragment comprises nivolumab, cemiplimab, pembrolizumab, avelumab, atezolizumab, or durvalumab.

12. The method of claim 11, wherein the antibody or antibody fragment is partially humanized, fully humanized, or chimeric.

13. The method of claim 10, wherein the PD-1 inhibitor comprises a small molecule, and wherein the small molecule comprises ibrutinib.

14. The method of claim 1, wherein the subject is human.

15. The method of claim 1, wherein the reference sample is obtained from healthy normal tissue, cancer that received a clinical benefit from PD-1 inhibition, or cancer that did not receive a clinical benefit from PD-1 inhibition.

16. The method of claim 1, wherein the reference sample is obtained from healthy normal tissue from the same individual as the test sample or one or more healthy normal tissues from different individuals.

17. The method of claim 1, wherein the expression level of the RT-associated gene is detected via a Gene Hybridization Array, or a real time reverse transcriptase polymerase chain reaction (real time RT-PCR) assay.

18. The method of claim 6, wherein the chemotherapeutic agent comprises thalidomide, lenalidomide, ibrutinib, ixazomib, bortezomib, carfilzomib, melphalan, vincristine, cyclophosphamide, doxorubicin, liposomal doxorubicin, or bendamustine.

19. The method of claim 1, wherein the expression level of the RT-associated gene is detected via hybridization-based analysis or polynucleotide sequencing.

20. The method of claim 1, wherein the RT-associated gene comprises CD226; and
determining that inhibition of PD-1 in the subject with RT will result in clinical benefit in the subject if the expression level of the CD226 gene in the test sample is higher than the level of the CD226 gene in the reference sample.
